(19) United States
(12) Patent Application Publication
(10) Pub. No.: US 2020/0392239 A1
(43) Pub. Date: Dec. 17, 2020

(54) USE OF A MULTIMERIC ANTI-DR5 BINDING MOLECULE IN COMBINATION WITH A CHEMOTHERAPEUTIC AGENT FOR TREATING CANCER

(71) Applicant: IGM Biosciences, Inc., Mountain View, CA (US)

(72) Inventors: Beatrice Tien-Yi WANG, Mountain View, CA (US); Bruce Alan KEYT, Hillsborough, CA (US)

(21) Appl. No.: 16/975,438

(22) PCT Filed: Feb. 25, 2019
(86) PCT No.: PCT/US2019/019340

§ 371 Date: Aug. 25, 2020

Related U.S. Application Data
(60) Provisional application No. 62/767,900, filed on Nov. 15, 2018.
(60) Provisional application No. 62/635,033, filed on Feb. 26, 2018.

(51) Int. Cl. C07K 16/28; A61P 35/00; A61K 31/437; A61K 31/7068; A61K 31/496

(52) U.S. Cl. CPC C07K 16/2878; A61P 35/00; A61K 31/496; A61K 31/7068; A61K 31/437

(57) ABSTRACT

This disclosure provides therapeutic methods for treating cancer including combination therapy with a multimeric anti-DR5 antibody and a chemotherapeutic agent, e.g., a type I topoisomerase inhibitor, a nucleoside analog, or a pro-apoptotic agent, e.g., a BCL-2 inhibitor.

### CROSS-REFERENCE TO RELATED APPLICATIONS

This application claims the benefit of U.S. Provisional Patent Application Ser. No. 62/635,033, filed Feb. 26, 2018 and U.S. Provisional Patent Application Ser. No. 62/767,900, filed Nov. 15, 2018, which are each incorporated herein by reference in their entireties.

### REFERENCE TO SEQUENCE LISTING SUBMITTED ELECTRONICALLY

The content of the electronically submitted sequence listing in ASCII text file (Name “09789-011WO1-Sequence-Listing; Size: 151,552 bytes; and Date of Creation: Feb. 11, 2019”) filed with the application is incorporated herein by reference in its entirety.

### BACKGROUND

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., PCT Publication Nos. WO 2015/153912, WO 2016/118641, WO 2016/141303, WO 2016/154593, WO 2016/168758, WO 2017/059387, WO 2017 059380, WO 2018/017888, WO 2018/017763, WO 2018/017889, and WO 2018/017761, the contents of which are incorporated herein by reference in their entireties.

Multimeric IgA or IgM antibodies present a useful tool for application to specific biological systems in which multiple components necessarily must be bound simultaneously to transmit biological signals. For instance, many receptor proteins on the surface of eukaryotic cells require the simultaneous activation of multiple monomers or subunits to achieve activation and transmission of a biological signal across a cell membrane, to the cytoplasm of the cell.

One such receptor is the apoptosis-inducing Tumor Necrosis Factor (TNF) receptor superfamily proteins DR5 (also referred to as TRAILR2). DR5 activation requires that at least three non-interacting receptor monomers be cross-linked, e.g., by a TRAIL ligand or agonist antibody, to form a stabilized receptor trimer, resulting in signal transduction across the cell membrane. Clustering of DR5 protein trimers into “rafts” of trimers can lead to more effective activation the signaling cascade.

Interest-in DR5 is heightened due to the finding that it is expressed in bladder cancer (Li et al., Urology, 79(4):968.e7-15, (2012)), gastric cancer (Lim et al., Carcinogen., 32(5):723-732, (2011)), ovarian cancer (Jiang et al., Mol. Med. Rep., 6(2):316-320, (2012)), pancreatic ductal adenocarcinoma (Rajeshkumar et al., Mol. Cancer Ther., 9(9):2583-92, (2010)), oral squamous cell carcinoma (Chen et al. Oncotarget 4:206-217, (2013)) and non-small cell lung cancer (Reck et al., Lung Canc., 82(3):441-448, (2013)). The current standard of care for certain of these cancers includes chemotherapeutic agents that disrupt cellular growth and metabolism, e.g., by blocking DNA synthesis, blocking cell division, or promoting apoptosis.

While certain anti-DR5 monoclonal antibodies, such as Tigatuzumab (CS-1008, Daiichi Sankyo Co. Ltd., disclosed in U.S. Pat. No. 7,244,429), have been found to be effective in vitro and in vivo even without additional cross-linkers added, these antibodies have not resulted in significant clinical efficacy. (See, Reck et al., 2013). More recently though, several different anti-DR5 IgM antibodies have been shown to have much higher efficacy both in vitro and in vivo. See, e.g., U.S. Patent Appl. Publication No. 2018-0009897, which is incorporated herein by reference in its entirety.

Better therapies and enhancements to existing therapies for difficult to treat tumors are needed, including combination therapies with anti-DR5 IgM antibodies.

### SUMMARY

This disclosure provides a method for inhibiting, delaying, or reducing malignant cell growth in a subject with cancer, where the method includes administering to a subject in need of treatment a combination therapy that includes an effective amount of a dimeric IgA antibody or a hexameric or pentameric IgM antibody, or a multimerized, antigen-binding fragment, variant, or derivative thereof that specifically and agonistically binds to DR5, wherein at least three antigen binding domains of the IgA or IgM antibody or fragment thereof are DR5-specific and agonistic; and an effective amount of a chemotherapeutic agent, e.g., a DNA topoisomerase I inhibitor, a nucleoside analog, or a pro-apoptotic agent, e.g., a BCL-2 inhibitor.

In certain aspects, at least three, at least four, at least five, at least six, at least seven, at least eight, at least nine, at least ten, at least eleven, or twelve antigen-binding domains of the antibody or fragment, variant, or derivative thereof can include a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 include the CDRs of an antibody comprising the VH and VL amino acid sequences SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73 or the six CDRs with one or two amino acid substitutions in one or more of the CDRs.

In certain aspects at least three, at least four, at least five, at least six, at least seven, at least eight, at least nine, at least ten, at least eleven, or twelve antigen-binding domains of the antibody or fragment, variant, or derivative thereof can include an antibody VH and a VL, wherein the VH and VL comprise amino acid sequences at least 90% identical to SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or wherein the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

In certain aspects, at least four, at least ten, or twelve antigen-binding domains of the antibody or fragment, variant, or derivative thereof can include antibody VH and VL regions with the amino acid sequences SEQ ID NO: 1 and SEQ ID NO: 2, SEQ ID NO: 5 and SEQ ID NO: 6, SEQ ID NO: 7 and SEQ ID NO: 8, SEQ ID NO: 84 and SEQ ID NO: 85, or SEQ ID NO: 88 and SEQ ID NO: 89, respectively.

In certain aspects the chemotherapeutic agent is a DNA topoisomerase I inhibitor.

In certain aspects the DNA topoisomerase I inhibitor is a camptothecin derivative or an active variant, isomer, or salt thereof. For example, the topoisomerase I inhibitor can be Irinotecan or Topotecan.

In certain aspects the chemotherapeutic agent is a nucleoside analog. In certain aspects the nucleoside analog is Gemcitabine.

In certain aspects the chemotherapeutic agent is a pro-apoptotic agent, e.g., a BCL-2 inhibitor, e.g., Venetoclax (ABT-199).

In certain aspects administration of the combination therapy can result in enhanced therapeutic efficacy relative to administration of the antibody or fragment, variant or derivative thereof or the chemotherapeutic agent, e.g., the DNA topoisomerase I inhibitor, the nucleoside analog, or the pro-apoptotic agent, e.g., the BCL-2 inhibitor, alone, such as, e.g., reduced tumor growth rate, tumor regression, or increased survival. In certain aspects the subject to be treated is a human subject.

### BRIEF DESCRIPTION OF THE DRAWINGS/FIGURES

FIG. 1A and FIG. 1B: Anti-DR5 IgM and Irinotecan combination induces more complete tumor cytotoxicity in vitro. FIG. 1A: HCT15 tumor cells treated with Anti-DR5 IgM (filled squares) or Anti-DR5 IgM plus 0.4 μM of Irinotecan (open squares). FIG. 1B: HCT15 tumor cells treated with Irinotecan (filled circles) or Irinotecan plus 1 ng/mL of Anti-DR5 IgM (open circles).

FIG. 2A and FIG. 2B: IgM efficacy in IgG resistant colon tumor model. FIG. 2A: Tumor volume in athymic nude mice implanted with IgG resistant HCT15 tumor cells and dosed with vehicle 5 times daily (crosses); 3 mg/kg of anti-DR5 IgG 3 times weekly (circles); 3 mg/kg of anti-DR5 IgM 5 times daily (squares); or 80 mg/kg of Irinotecan 3 times within the first week (triangles). FIG. 2B: Overall survival of athymic nude mice implanted with IgG resistant HCT15 tumor cells and dosed with vehicle 5 times daily (solid black line), 3 mg/kg of anti-DR5 IgG 3 times weekly (dashed black line), 3 mg/kg of anti-DR5 IgM 5 times daily (dashed gray line), or 80 mg/kg of Irinotecan 3 times within the first week (dotted black line).

FIG. 3A and FIG. 3B: No enhanced efficacy combining anti-DR5 IgG with Irinotecan standard of care. FIG. 3A: Tumor volume in athymic nude mice implanted with IgG resistant HCT15 tumor cells and dosed with vehicle 5 times daily (crosses); 3 mg/kg of anti-DR5 IgG 3 times weekly (filled circles); 80 mg/kg of Irinotecan 3 times within the first week (filled triangles); or the combined anti-DR5 IgG and Irinotecan dosing regimens (open circles). FIG. 3B: Overall survival of athymic nude mice implanted with IgG resistant HCT15 tumor cells and dosed with vehicle 5 times daily (solid black line), 3 mg/kg of anti-DR5 IgG 3 times weekly (dashed black line), 80 mg/kg of Irinotecan 3 times within the first week (dotted black line), or the combined anti-DR5 IgG and Irinotecan dosing regimens (solid gray line).

FIG. 4A and FIG. 4B: Significantly enhanced efficacy combining anti-DR5 IgM with Irinotecan standard of care. FIG. 4A: Tumor volume in athymic nude mice implanted with IgG resistant HCT15 tumor cells and dosed with vehicle 5 times daily (crosses); 3 mg/kg of anti-DR5 IgM 5 times daily (squares); 80 mg/kg of Irinotecan 3 times within the first week (filled triangles); or the combined anti-DR5 IgM and Irinotecan dosing regimens (open squares). FIG. 4B: Overall survival of athymic nude mice implanted with IgG resistant HCT15 tumor cells and dosed with vehicle 5 times daily (solid black line), 3 mg/kg of anti-DR5 IgM 5 times daily (dashed gray line), 80 mg/kg of Irinotecan 3 times within the first week (dotted black line), or the combined anti-DR5 IgM and Irinotecan dosing regimens (solid gray line).

FIG. 5A and FIG. 5B: Anti-DR5 IgM and Gemcitabine combination induces more complete tumor cytotoxicity in vitro. FIG. 5A: BxPC3 pancreatic tumor cells were treated with 0.56 μM of Gemcitabine (black bar), 4 ng/mL of anti-DR5 IgM Mab #5 (gray bar), or a combination of the two agents (white bar). FIG. 5B: Panc-1 pancreatic tumor cells were treated with 0.56 μM of Gemcitabine (black bar), 4 ng/mL of anti-DR5 IgM Mab #5 (gray bar), or a combination of the two agents (white bar).

FIG. 6A and FIG. 6B: Anti-DR5 IgM and Gemcitabine combination induces more complete tumor cytotoxicity in vitro. FIG. 6A: BxPC3 pancreatic tumor cells were treated with serial dilutions of Anti-DR5 IgM Mab #5 alone (open circles) or in combination with 0.06 μM of Gemcitabine (filled diamonds), 0.19 μM of Gemcitabine (inverted filled triangles), 0.56 μM of Gemcitabine (upright filled triangles), 1.67 μM of Gemcitabine (filled squares), or 5 μM of Gemcitabine (filled circles). FIG. 6B: Panc-1 pancreatic tumor cells were treated with serial dilutions of Anti-DR5 IgM Mab #5 alone (open circles) or in combination with 0.06 μM of Gemcitabine (filled diamonds), 0.19 μM of Gemcitabine (inverted filled triangles), 0.56 μM of Gemcitabine (upright filled triangles), 1.67 μM of Gemcitabine (filled squares), or 5 μM of Gemcitabine (filled circles).

FIG. 7A and FIG. 7B: Weakly enhanced efficacy combining anti-DR5 IgG with Gemcitabine standard of care. FIG. 7A: Tumor volume in nude mice implanted subcutaneously with BxPC3 pancreatic tumor fragments and dosed with vehicle 7 times daily (crosses); a single 3 mg/kg dose of anti-DR5 IgG Mab #2 (filled circles); 120 mg/kg of Gemcitabine every 3 days for a total of 4 doses (filled triangles); or the combined anti-DR5 IgG and Gemcitabine dosing regimens (open circles). FIG. 7B: Overall survival of nude mice implanted subcutaneously with BxPC3 pancreatic tumor fragments and dosed with vehicle 7 times daily (solid black line); a single 3 mg/kg dose of anti-DR5 IgG Mab #2 (dashed black line); 120 mg/kg of Gemcitabine every 3 days for a total of 4 doses (dotted black line); or the combined anti-DR5 IgG and Gemcitabine dosing regimens (gray solid line).

FIG. 8A and FIG. 8B: Enhanced tumor efficacy combining anti-DR5 IgM with Gemcitabine standard of care. FIG. 8A: Tumor volume in nude mice implanted subcutaneously with BxPC3 pancreatic tumor fragments and dosed with vehicle 7 times daily (crosses); 3 mg/kg of anti-DR5 IgM Mab #2 7 times daily (filled squares); 120 mg/kg of Gemcitabine every 3 days for a total of 4 doses (filled triangles); or the combined anti-DR5 IgM and Gemcitabine dosing regimens (open squares). FIG. 8B: Overall survival of nude mice implanted subcutaneously with BxPC3 pancreatic tumor fragments and dosed with vehicle 7 times daily (solid black line); 3 mg/kg of anti-DR5 IgM Mab #2 7 times daily (dashed gray line); 120 mg/kg of Gemcitabine every 3 days for a total of 4 doses (dotted black line); or the combined anti-DR5 IgM and Gemcitabine dosing regimens (gray solid line).

FIG. 9A and FIG. 9B: Anti-DR5 IgM and Venetoclax combination induces more complete tumor cytotoxicity in vitro. FIG. 9A: Molm-13 AML tumor cells were treated with 1.2 ng/mL of anti-DR5 IgM Mab #5 (gray bar), 3.7 nM of Venetoclax (black bar), or a combination of the two agents (white bar). FIG. 9B: MV-4-11 AML tumor cells were treated with 37 ng/mL of anti-DR5 IgM Mab #5 (gray bar), 3.7 nM of Venetoclax (black bar), or a combination of the two agents (white bar).

FIG. 10A and FIG. 10B: Anti-DR5 IgM and Venetoclax combination induces more complete tumor cytotoxicity in vitro. FIG. 10A: Molm-13 AML tumor cells were treated with serial dilutions of Anti-DR5 IgM Mab #5 alone (open circles) or in combination with 1.2 nM of Venetoclax (inverted filled triangles), 3.7 nM of Venetoclax (upright filled triangles), 11 nM of Venetoclax (filled squares), or 33 nM of Venetoclax (filled circles). FIG. 10B: MV-4-11 AML tumor cells were treated with serial dilutions of Anti-DR5 IgM Mab #5 alone (open circles) or in combination with 1.2 nM of Venetoclax (inverted filled triangles), 3.7 nM of Venetoclax (upright filled triangles), 11 nM of Venetoclax (filled squares), 33 nM of Venetoclax (filled circles), or 100 nM of Venetoclax (open squares).

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

As used herein, the terms “DR5” or “TRAILR2” refer to a member of the family of Tumor Necrosis Factor transmembrane receptor proteins expressed on the surface of various cells and tissues, which, upon activation, can induce apoptosis of the cell.

Disclosed herein are certain binding molecules, or antigen-binding fragments, variants, or derivatives thereof that bind to DR5, thereby eliciting cellular apoptosis. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold. Where a binding molecule is a polymeric binding molecule, e.g., a pentameric or hexameric IgM antibody or a dimeric IgA antibody, it is understood when referring to multimeric fragments, variants, or derivatives, that the fragment, variant, or derivative continues to be multimeric.

As used herein, the term “binding molecule” refers in its broadest sense to a molecule that specifically binds to a receptor, e.g., an epitope or an antigenic determinant. As described further herein, a binding molecule can comprise one of more “antigen binding domains” described herein. A non-limiting example of a binding molecule is an antibody or fragment thereof that retains antigen-specific binding.

As used herein, the terms “binding domain” or “antigen binding domain” refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.” Other binding domains include, without limitation, the variable heavy chain (VHH) of an antibody derived from a camelid species, or six immunoglobulin complementarity determining regions (CDRs) expressed in a fibronectin scaffold. A “binding molecule” as described herein can include one, two, three, four, five, six, seven, eight, nine, ten, eleven, twelve or more “antigen binding domains.”

The terms “antibody” and “immunoglobulin” can be used interchangeably herein. An antibody (or an antigen-binding fragment, variant, or derivative thereof, or a multimeric fragment, variant, or derivative thereof, as disclosed herein) includes at least the variable domain of a heavy chain (for camelid species) or at least the variable domains of a heavy chain and a light chain, and for a multimeric molecule, at least the Cμ4-tp or Cα3-tp constant region domains to allow multimerization. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, a dimeric IgA antibody that includes four complete heavy chains and four complete light chains and includes a J chain and/or a secretory component, or a pentameric or hexameric IgM antibody that includes ten or twelve complete heavy chains and ten or twelve complete light chains and optionally includes a J chain.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody or antigen-binding fragment thereof, which corresponds to a standard “H2L2” immunoglobulin structure, i.e., two heavy chains or fragments thereof and two light chains or fragments thereof. In certain aspects, e.g., where the binding molecule is a bivalent IgG antibody or antigen-binding fragment thereof, the terms “binding molecule” and “binding unit” are equivalent. In other aspects, e.g., where the binding molecule is an IgA dimer, an IgM pentamer, or an IgM hexamer, the binding molecule comprises two, five, or six “binding units,” respectively. A binding unit need not include full-length antibody heavy and light chains, but will typically be bivalent, i.e., will include two “binding domains,” as defined herein. Certain binding molecules provided in this disclosure are dimeric and include two bivalent binding units that include IgA constant regions or fragments thereof. Certain binding molecules provided in this disclosure are pentameric or hexameric and include five or six bivalent binding units that include IgM constant regions or the necessary fragments thereof to allow multimerization. A binding molecule comprising two or more, e.g., two, five, or six binding units, is referred to herein as “multimeric.”

The terms “valency,” “bivalent,” “multivalent” and grammatical equivalents, refer to the number of binding domains in given binding molecule or binding unit. As such, the terms “bivalent”, “tetravalent”, and “hexavalent” in reference to a given binding molecule, e.g., an IgM antibody or fragment thereof, denote the presence of two binding domains, four binding domains, and six binding domains, respectively. In a typical IgM-derived binding molecule where each binding unit is bivalent, the binding molecule itself can have 10 or 12 valencies. A bivalent or multivalent binding molecule can be monospecific, i.e., all of the binding domains are the same, or can be bispecific or multispecific, e.g., where two or more binding domains are different, e.g., bind to different epitopes on the same antigen, or bind to entirely different antigens.

The term “epitope” includes any molecular determinant capable of specific binding to an antibody. In certain aspects, an epitope can include chemically active surface groupings of molecules such as amino acids, sugar side chains, phosphoryl, or sulfonyl, and, in certain aspects, can have a three-dimensional structural characteristics, and or specific charge characteristics. An epitope is a region of a target that is bound by an antibody.

The term “target” is used in the broadest sense to include substances that can be bound by a binding molecule. A target can be, e.g., a polypeptide, a nucleic acid, a carbohydrate, a lipid, or other molecule. Moreover, a “target” can, for example, be a cell, an organ, or an organism that comprises an epitope bound that can be bound by a binding molecule.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (e.g., CH1, CH2, CH3, or CH4 (where present)) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4-tp in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody constant heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody constant heavy chain constant domain 1 (CA1 or Cα1), an antibody heavy chain constant domain 2 (CA2 or Cα2), and an antibody heavy chain constant domain 3 (CA3 or Cα3) that can include a tailpiece.

As indicated above, variable regions allow a binding molecule to selectively recognize and specifically bind epitopes on antigens. That is, the VL domain and VH domain, or subset of the complementarity determining regions (CDRs), of a binding molecule, e.g., an antibody, combine to form the antigen binding domain. More specifically, an antigen binding domain can be defined by three CDRs on each of the VH and VL chains. Certain antibodies form larger structures. For example, IgA can form a molecule that includes two H2L2 binding units and a J chain covalently connected via disulfide bonds, which can be further associated with a secretory component, and IgM can form a pentameric or hexameric molecule that includes five or six H2L2 binding units and optionally a J chain covalently connected via disulfide bonds.

The six “complementarity determining regions” or “CDRs” present in an antibody antigen-binding domain are short, non-contiguous sequences of amino acids that are specifically positioned to form the binding domain as the antibody assumes its three-dimensional configuration in an aqueous environment. The remainder of the amino acids in the binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (www://imgt.cines.fr/) (IMGT®/V-Quest) to identify variable region segments, including CDRs. (See, e.g., Brochet et al., Nucl. Acids Res., 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof, or multimeric fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of binding domains and an antigen. See, e.g., Harlow at pages 29-34. Avidity is related to both the affinity of individual binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶ M, 10⁻⁶M, 5×10⁻⁷M, 10⁻⁷M, 5×10⁻⁸M, 10⁻⁸M, 5×10⁻⁹ M, 10⁻⁹ M, 5×10⁻¹° M, 10⁻¹° M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵ M, or 10⁻¹⁵M.

Antibody fragments including single-chain antibodies or other binding domains can exist alone or in combination with one or more of the following: hinge region, CH1, CH2, CH3, or CH4-tp domains, J chain, or secretory component. Also included are antigen-binding fragments that can include any combination of variable region(s) with one or more of a hinge region, CH1, CH2, CH3, or CH4 domains, a J chain, or a secretory component, e.g., to allow multimerization. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al.

As used herein the term “subunit” refers to a single polypeptide chain that combines with other identical or heterologous polypeptide chains to produce a binding molecule, e.g., an antibody or antigen-binding fragment thereof.

As used herein, the term “heavy chain subunit” includes amino acid sequences derived from an immunoglobulin heavy chain, a binding molecule, e.g., an antibody comprising a heavy chain subunit can include at least one of: a VH domain, a CH1 domain, a hinge (e.g., upper, middle, and/or lower hinge region) domain, a CH2 domain, a CH3 domain, a CH4-tp domain, or a variant or fragment thereof. For example, a binding molecule, e.g., an antibody or fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain aspects a binding molecule, e.g., an antibody or fragment, variant, or derivative thereof can include, in addition to a VH domain, a CH3 domain and a CH4-tp domain; or a CH3 domain, a CH4-tp domain, and a J chain. The constant region portions can in some instances be from the same isotype, e.g., all Cμ constant domains, or they can be a mixture, e.g., some of the constant domains can be Cμ constant domains (e.g., the Cμ.4-tp domain) while other constant domains can be from another antibody isotype (e.g., a Cy2 and Cy3 constant domain). Further, a binding molecule for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As previously indicated, the structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical IgG heavy chain molecule.

As used herein the term “CH2 domain” includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat E A et al., op. cit. The CH3 domain extends from the CH2 domain to the C-terminal of the IgG molecule and comprises approximately 108 amino acids. Certain immunoglobulin classes, e.g., IgM, further include a CH4-tp region.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody that has binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, I Drugs. 13:543-9 (2010)). A bispecific antibody can also be a diabody.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

As used herein, the term “cross-linked” refers to joining together of two or more molecules by a third molecule. For example, a bivalent antibody with two binding domains that specifically bind to the same antigen can “cross-link” two copies of that antigen, e.g., as they are expressed on a cell. Many TNF superfamily receptor proteins, including DR5, require cross-linking of three or more receptors on the surface of a cell for activation. Cross-linking of DR5 proteins means, for instance, contacting a binding molecule, as disclosed herein, with DR5 expressed on the surface of a cell such that at least three DR5 monomers are simultaneously bound together by one or more binding molecules, thereby activating the receptors.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “cancer” and “cancerous” refer to or describe the physiological condition in mammals in which a population of cells are characterized by unregulated cell growth. Cancers can be categorized, e.g., as solid tumors or malignancies, or hematological cancers or malignancies. Both types can migrate to remote sites as metastases. A solid tumor can be categorized, e.g., as a sarcoma, a carcinoma, a melanoma, or a metastasis thereof.

The terms “proliferative disorder” and “proliferative disease” refer to disorders associated with abnormal cell proliferation such as cancer. “Tumor” and “neoplasm” as used herein refer to any mass of tissue that result from excessive cell growth or proliferation, either benign (noncancerous) or malignant (cancerous) including pre-cancerous lesions.

The terms “metastasis,” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site.

Examples of such solid tumors can include, e.g., squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer (NSCLC), adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, prostate cancer, vulval cancer, thyroid cancer, head and neck cancer, any metastases thereof, or any combination thereof.

Examples of hematologic cancers or malignancies include without limitation leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain embodiments, cancers that are amenable to treatment via the methods provided herein include, but are not limited to sarcomas, breast carcinomas, ovarian cancer, cervical cancer, head and neck cancer, NSCLC, esophageal cancer, gastric cancer, kidney cancer, liver cancer, bladder cancer, colorectal cancer, and pancreatic cancer.

The term “therapeutically effective amount” refers to an amount of an antibody, polypeptide, polynucleotide, small organic molecule, or other drug effective to “treat” or in some instances, “prevent” a disease or disorder in a subject, e.g., a human. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. A subject is successfully “treated” according to the methods of the present disclosure if the patient shows one or more of the following: a reduction in the number of or complete absence of cancer cells; a reduction in the tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” includes subjects, such as mammalian subjects, that would benefit from administration of a binding molecule such as an antibody, comprising one or more antigen binding domains. Such binding molecules, e.g., antibodies, can be used, e.g., for a diagnostic procedures and/or for treatment or prevention of a disease.

IgM Binding Molecules

IgM is the first immunoglobulin produced by B cells in response to stimulation by antigen and is present at around 1.5 mg/ml in serum with a half-life of 5 days. IgM is a pentameric or hexameric molecule. An IgM binding unit includes two light and two heavy chains. While IgG contains three heavy chain constant domains (CH1, CH2 and CH3), the heavy (μ) chain of IgM additionally contains a fourth constant domain (CH4), that includes a C-terminal “tailpiece” (tp). The human IgM constant region typically comprises the amino acid sequence SEQ ID NO: 74. The human CO region ranges from about amino acid 5 to about amino acid 102 of SEQ ID NO: 74; the human Cμ2 region ranges from about amino acid 114 to about amino acid 205 of SEQ ID NO: 74, the human Cμ3 region ranges from about amino acid 224 to about amino acid 319 of SEQ ID NO: 74, the Cμ4 region ranges from about amino acid 329 to about amino acid 430 of SEQ ID NO: 74, and the tailpiece ranges from about amino acid 431 to about amino acid 453 of SEQ ID NO: 74. SEQ ID NO: 74 is presented below:

GSASAPTLFPLVSCENSPSDTSSVAVGCLAQDFLPDSITL
SWKYKNNSDISSTRGFPSVLRGGKYAATSQVLLPSKDV
MQGTDEHVVCKVQHPNGNKEKNVPLPVIAELPPKVSVF
VPPRDGFFGNPRKSKLICQATGFSPRQIQVSWLREGKQV
GSGVTTDQVQAEAKESGPTTYKVTSTLTIKESDWLGQS
MFTCRVDHRGLTFQQNASSMCVPDQDTAIRVFAIPPSFA
SIFLTKSTKLTCLVTDLTTYDSVTISWTRQNGEAVKTHT
NISESHPNATFSAVGEASICEDDWNSGERFTCTVTHTDLP
SPLKQTISRPKGVALHRPDVYLLPPAREQLNLRESATITC
LVTGFSPADVFVQWMQRGQPLSPEKYVTSAPMPEPQAP
GRYFAHSILTVSEEEWNTGETYTCVAHEALPNRVTERTV
DKSTGKPTLYNVSLVMSDTAGTCY

Five IgM binding units can form a complex with an additional small polypeptide chain (the J chain) to form an IgM antibody. The mature human J chain comprises the amino acid sequence SEQ ID NO: 76. Without the J chain, IgM binding units typically assemble into a hexamer. While not wishing to be bound by theory, the assembly of IgM binding units into a pentameric or hexameric binding molecule is thought to involve the Cμ3 and Cμ4 domains. Accordingly, a pentameric or hexameric binding molecule provided in this disclosure typically includes IgM constant regions that include at least the Cμ3 and Cμ.4 domains. SEQ ID NO: 76 is presented below:

QEDERIVLVDNKCKCARITSRIIRSSEDPNEDIVERNIRIIV
PLNNRENISDPTSPLRTRFVYHLSDLCKKCDPTEVELDN
QIVTATQSNICDEDSATETCYTYDRNKCYTAVVPLVYG
GETKMVETALTPDACYPD

An IgM heavy chain constant region can additionally include a Cμ2 domain or a fragment thereof, a Cμ1 domain or a fragment thereof, and/or other IgM heavy chain domains. In certain aspects, a binding molecule as provided herein can include a complete IgM heavy (μ) chain constant domain, e.g., SEQ ID NO: 74, or a variant, derivative, or analog thereof.

Pentameric or Hexameric anti-DR5 Binding Molecules

This disclosure provides a pentameric or hexameric binding molecule, i.e., a binding molecule with five or six “binding units” as defined herein, that can specifically bind to DR5. A binding molecule as provided herein can possess improved binding characteristics or biological activity as compared to a binding molecule composed of a single binding unit, e.g., a bivalent IgG antibody. For example, a pentameric or hexameric binding molecule can more efficiently cross-link three or more DR5 molecules on the surface of a cell, e.g., a tumor cell, thereby facilitating apoptosis of the cell.

A binding molecule as provided herein can likewise possess distinctive characteristics compared to multivalent binding molecule composed of synthetic or chimeric structures. For example, use of human IgM constant regions can afford reduced immunogenicity and thus increased safety relative to a binding molecule containing chimeric constant regions or synthetic structures. Moreover, an IgM-based binding molecule can consistently form hexameric or pentameric oligomers resulting in a more homogeneous expression product. Superior complement fixation can also be an advantageous effector function of IgM-based binding molecules.

In certain aspects, the disclosure provides a pentameric or hexameric binding molecule comprising five or six bivalent binding units, respectively, where each binding unit includes two IgM heavy chain constant regions or fragments thereof. In certain aspects, the two IgM heavy chain constant regions are human heavy chain constant regions.

Where the binding molecule provided herein is pentameric, the binding molecule can further comprise a J chain, or fragment thereof, or variant thereof.

An IgM heavy chain constant region can include one or more of a Cμ1 domain, a Cμ2 domain, a Cμ3 domain, and/or a Cμ4 domain, provided that the constant region can serve a desired function in the binding molecule, e.g., associate with second IgM constant region to form a binding domain, or associate with other binding units to form a hexamer or a pentamer. In certain aspects the two IgM heavy chain constant regions or fragments thereof within an individual binding unit each comprise a Cμ3 domain or fragment thereof, a Cμ4 domain or fragment thereof, a tailpiece (TP) or fragment thereof, or any combination of a Cμ3 domain a Cμ domain, and a TP or fragment thereof. In certain aspects the two IgM heavy chain constant regions or fragments thereof within an individual binding unit each further comprise a Cμ2 domain or fragment thereof, a CO domain or fragment thereof, or a Cμ1 domain or fragment thereof and a Cμ2 domain or fragment thereof.

In certain aspects each of the two IgM heavy chain constant regions in a given binding unit is associated with an antigen-binding domain, for example an Fv portion of an antibody, e.g., a VH and a VL of a human or murine antibody, where the VL can be associated with a light chain constant region. In a binding molecule as provided herein at least three antigen-binding domains of the binding molecule are DR5 binding domains, i.e., binding domains that can specifically bind to DR5, e.g., human DR5.

IgA Binding Molecules

IgA plays a critical role in mucosal immunity and comprises about 15% of total immunoglobulin produced. IgA is a monomeric or dimeric molecule. An IgA binding unit includes two light and two heavy chains. IgA contains three heavy chain constant domains (Cα1, Cα2 and Cα3), and includes a C-terminal “tailpiece.” Human IgA has two subtypes, IgA1 and IgA2. The human IgA1 constant region typically comprises the amino acid sequence SEQ ID NO: 78. The human Cα1 region ranges from about amino acid 6 to about amino acid 98 of SEQ ID NO: 78; the human Cα2 region ranges from about amino acid 125 to about amino acid 220 of SEQ ID NO: 78, the human Cα3 region ranges from about amino acid 228 to about amino acid 330 of SEQ ID NO: 78, and the tailpiece ranges from about amino acid 331 to about amino acid 352 of SEQ ID NO: 78. The human IgA2 constant region typically comprises the amino acid sequence SEQ ID NO: 79. The human Cal region ranges from about amino acid 6 to about amino acid 98 of SEQ ID NO: 79; the human Cα2 region ranges from about amino acid 112 to about amino acid 207 of SEQ ID NO: 79, the human Cα3 region ranges from about amino acid 215 to about amino acid 317 of SEQ ID NO: 79, and the tailpiece ranges from about amino acid 318 to about amino acid 340 of SEQ ID NO: 79. SEQ ID NOS: 78 and 79 are presented below:

SEQ ID NO: 78
ASPTSPKVFPLSLCSTQPDGNVVIACLVQGFFPQEPLSVT
WSESGQGVTARNFPPSQDASGDLYTTSSQLTLPATQCLA
GKSVTCHVKHYTNPSQDVTVPCPVPSTPPTPSPSTPPTPS
PSCCHPRLSLHRPALEDLLLGSEANLTCTLTGLRDASGV
TFTWTPSSGKSAVQGPPERDLCGCYSVSSVLPGCAEPW
NHGKTFTCTAAYPESKTPLTATLSKSGNTFRPEVHLLPPP
SEELALNELVTLTCLARGFSPKDVLVRWLQGSQELPREK
YLTWASRQEPSQGTTTFAVTSILRVAAEDWKKGDTFSC
MVGHEALPLAFTQKTIDRLAGKPTHVNVSVVMAEVDG
TCY
SEQ ID NO: 79
ASPTSPKVFPLSLDSTPQDGNVVVACLVQGFFPQEPLSV
TWSESGQNVTARNFPPSQDASGDLYTTSSQLTLPATQCP
DGKSVTCHVKHYTNPSQDVTVPCPVPPPPPCCHPRLSLH
RPALEDLLLGSEANLTCTLTGLRDASGATFTWTPSSGKS
AVQGPPERDLCGCYSVSSVLPGCAQPWNHGETFTCTAA
HPELKTPLTANITKSGNTFRPEVHLLPPPSEELALNELVT
LTCLARGFSPKDVLVRWLQGSQELPREKYLTWASRQEP
SQGTTTFAVTSILRVAAEDWKKGDTFSCMVGHEALPLA
FTQKTIDRMAGKPTHVNVSVVMAEVDGTCY

Two IgA binding units can form a complex with two additional polypeptide chains, the J chain (SEQ ID NO: 76) and the secretory component (precursor, SEQ ID NO: 80, mature, SEQ ID NO: 81) to form a secretory IgA (sIgA) antibody. While not wishing to be bound by theory, the assembly of IgA binding units into a dimeric sIgA binding molecule is thought to involve the Cα3 and tailpiece domains. Accordingly, a dimeric sIgA binding molecule provided in this disclosure typically includes IgA constant regions that include at least the Cα3 and tailpiece domains. SEQ ID NO: 80 and SEQ ID NO: 81 are presented below:

SEQ ID NO: 80:
MLLFVLTCLLAVFPAISTKSPIFGPEEVNSVEGNSVSITCY
YPPTSVNRHTRKYWCRQGARGGCITLISSEGYVSSKYAG
RANLTNFPENGTFVVNIAQLSQDDSGRYKCGLGINSRGL
SFDVSLEVSQGPGLLNDTKVYTVDLGRTVTINCPFKTEN
AQKRKSLYKQIGLYPVLVIDSSGYVNPNYTGRIRLDIQG
TGQLLFSVVINQLRLSDAGQYLCQAGDDSNSNKKNADL
QVLKPEPELVYEDLRGSVTFHCALGPEVANVAKFLCRQ
SSGENCDVVVNTLGKRAPAFEGRILLNPQDKDGSFSVVI
TGLRKEDAGRYLCGAHSDGQLQEGSPIQAWQLFVNEES
TIPRSPTVVKGVAGGSVAVLCPYNRKESKSIKYWCLWE
GAQNGRCPLLVDSEGWVKAQYEGRLSLLEEPGNGTFTV
ILNQLTSRDAGFYWCLTNGDTLWRTTVEIKIIEGEPNLK
VPGNVTAVLGETLKVPCHFPCKFSSYEKYWCKWNNTG
CQALPSQDEGPSKAFVNCDENSRLVSLTLNLVTRADEG
WYWCGVKQGHFYGETAAVYVAVEERKAAGSRDVSLA
KADAAPDEKVLDSGFREIENKAIQDPRLFAEEKAVADTR
DQADGSRASVDSGSSEEQGGSSRALVSTLVPLGLVLAV
GAVAVGVARARHRKNVDRVSIRSYRTDISMSDFENSRE
FGANDNMGASSITQETSLGGKEEFVATTESTTETKEPKK
AKRSSKEEAEMAYKDFLLQSSTVAAEAQDGPQEA
SEQ ID NO: 81:
KSPIFGPEEVNSVEGNSVSITCYYPPTSVNRHTRKYWCR
QGARGGCITLISSEGYVSSKYAGRANLTNFPENGTFVVNI
AQLSQDDSGRYKCGLGINSRGLSFDVSLEVSQGPGLLND
TKVYTVDLGRTVTINCPFKTENAQKRKSLYKQIGLYPVL
VIDSSGYVNPNYTGRIRLDIQGTGQLLFSVVINQLRLSDA
GQYLCQAGDDSNSNKKNADLQVLKPEPELVYEDLRGSV
TFHCALGPEVANVAKFLCRQSSGENCDVVVNTLGKRAP
AFEGRILLNPQDKDGSFSVVITGLRKEDAGRYLCGAHSD
GQLQEGSPIQAWQLFVNEESTIPRSPTVVKGVAGGSVAV
LCPYNRKESKSIKYWCLWEGAQNGRCPLLVDSEGWVK
AQYEGRLSLLEEPGNGTFTVILNQLTSRDAGFYWCLTNG
DTLWRTTVEIKIIEGEPNLKVPGNVTAVLGETLKVPCHFP
CKFSSYEKYWCKWNNTGCQALPSQDEGPSKAFVNCDE
NSRLVSLTLNLVTRADEGWYWCGVKQGHFYGETAAVY
VAVEERKAAGSRDVSLAKADAAPDEKVLDSGFREIENK
AIQDPR

An IgA heavy chain constant region can additionally include a Cα2 domain or a fragment thereof, a Cα1 domain or a fragment thereof, and/or other IgA heavy chain domains. In certain aspects, a binding molecule as provided herein can include a complete IgA heavy (α) chain constant domain (e.g., SEQ ID NO: 78 or SEQ ID NO: 79), or a variant, derivative, or analog thereof.

Dimeric DR5 Binding Molecules

This disclosure provides a dimeric binding molecule, e.g., a binding molecule with two IgA “binding units” or fragments, variants, or derivatives thereof as defined herein, that can specifically bind to DR5. A binding molecule as provided herein can possess improved binding characteristics or biological activity as compared to a binding molecule composed of a single binding unit, e.g., a bivalent IgG antibody. For example, an IgA binding molecule can more efficiently cross-link three or more DR5 monomers on the surface of a cell, e.g., a tumor cell, thereby facilitating apoptosis of the cell. Moreover, an IgA binding molecule can reach mucosal sites providing greater tissue distribution for the binding molecules provided herein. Use of an IgA-based binding molecule can allow, for example, greater tissue distribution for a binding molecule provided herein. Mucosal distribution could be beneficial for certain cancers, e.g., lung cancer, gastric cancer, ovarian cancer, colorectal cancer, or squamous cell carcinoma. Likewise, a dimeric binding molecule as provided herein can possess binding characteristics or biological activity that can be distinguished from a binding molecule comprising five or six binding units, e.g., a hexameric or pentameric IgM antibody. For example, a dimeric binding molecule would be smaller, and could, for example, achieve better tissue penetration in solid tumors.

In certain aspects, the disclosure provides a dimeric binding molecule comprising two bivalent binding units, where each binding unit includes two IgA heavy chain constant regions or fragments thereof. In certain aspects, the two IgA heavy chain constant regions are human heavy chain constant regions.

A dimeric IgA binding molecule as provided herein can further comprise a J chain, or fragment thereof, or variant thereof. A dimeric IgA binding molecule as provided herein can further comprise a secretory component, or fragment thereof, or variant thereof.

An IgA heavy chain constant region can include one or more of a Cα1 domain, a Cα2 domain, and/or a Cα3 domain, provided that the constant region can serve a desired function in the binding molecule, e.g., associate with a light chain constant region to facilitate formation of an antigen binding domain, or associate with another IgA binding unit to form a dimeric binding molecule. In certain aspects the two IgA heavy chain constant regions or fragments thereof within an individual binding unit each comprise a Cα3 domain or fragment thereof, a tailpiece (TP) or fragment thereof, or any combination of a Cα3 domain, a TP, or fragment thereof. In certain aspects the two IgA heavy chain constant regions or fragments thereof within an individual binding unit each further comprise a Cα2 domain or fragment thereof, a Cal domain or fragment thereof, or a Cal domain or fragment thereof and a Cα2 domain or fragment thereof.

In certain aspects each of the two IgA heavy chain constant regions in a given binding unit is associated with an antigen binding domain, for example an Fv portion of an antibody, e.g., a VH and a VL of a human or murine antibody, where the VL can be associated with a light chain constant region. In a binding molecule as provided herein at least three antigen-binding domains of the binding molecule are DR5 binding domains, i.e., binding domains that can specifically bind to DR5, e.g., human DR5.

Modified J Chains

In certain aspects, the J chain of dimeric or pentameric binding molecules as provided herein can be modified, e.g., by introduction of a heterologous moiety, or two or more heterologous moieties, without interfering with the ability of the IgM or IgA binding molecule to assemble and bind to its binding target(s). See PCT Publication No. WO 2015/153912, PCT Publication No. WO 2017/059387, and PCT Publication No. WO 2017/059380, each of which is incorporated herein by reference in its entirety. Accordingly, dimeric or pentameric binding molecules as provided herein, including multispecific dimeric or pentameric binding molecules as described elsewhere herein, can comprise a modified J chain or functional fragment thereof comprising a heterologous moiety introduced into the J chain or fragment thereof. In certain aspects heterologous moiety can be a peptide or polypeptide sequence fused in frame to the J chain or chemically conjugated to the J chain. In certain aspects the heterologous moiety can be a chemical moiety conjugated to the J chain. Heterologous moieties to be attached to a J chain can include, without limitation, a binding moiety, e.g., an antibody or antigen binding fragment thereof, e.g., a single chain Fv (ScFv) molecule, a stabilizing peptide that can increase the half-life of the dimeric or pentameric binding molecule, or a chemical moiety such as a polymer or a cytotoxin.

In some embodiments, a modified J chain can comprise an antigen binding domain that can include without limitation a polypeptide (including small peptides) capable of specifically binding to a target antigen. In certain aspects, an antigen binding domain associated with a modified J chain can be an antibody or an antigen-binding fragment thereof, as described elsewhere herein. In certain aspects the antigen binding domain can be a scFv binding domain or a single-chain binding domain derived, e.g., from a camelid or condricthoid antibody. The antigen binding domain can be introduced into the J chain at any location that allows the binding of the antigen binding domain to its binding target without interfering with J chain function or the function of an associated IgM or IgA antibody. Insertion locations include, but are not limited to, at or near the C-terminus, at or near the N-terminus or at an internal location that, based on the three-dimensional structure of the J chain, is accessible. In certain aspects, the antigen binding domain can be introduced into the human J chain of SEQ ID NO: 76 between cysteine residues 92 and 101 of SEQ ID NO: 76. In a further aspect, the antigen binding domain can be introduced into the human J chain of SEQ ID NO: 76 at or near a glycosylation site. In a further aspect, the antigen binding domain can be introduced into the human J chain of SEQ ID NO: 76 within about 10 amino acid residues from the C-terminus.

DR5 Binding Domains

A DR5 binding molecule, e.g., an anti-DR5 antibody or fragment, variant, or derivative thereof as provided herein can be dimeric, pentameric, or hexameric, comprising two, five, or six bivalent binding units, respectively. The binding units can be full length or variants or fragments thereof that retain binding function.

Each binding unit comprises two IgA or IgM heavy chain constant regions or fragments thereof, each associated with an antigen-binding domain. As noted above, an antigen binding domain is a region of a binding molecule that is necessary and sufficient to specifically bind to an epitope. A “binding molecule” as described herein can include one, two, three, four, five, six, seven, eight, nine, ten, eleven, twelve or more “antigen binding domains.”

A dimeric, pentameric, or hexameric binding molecule as provided herein can include at least three antigen-binding domains which specifically and agonistically bind to DR5. As noted above DR5, upon activation, can induce apoptosis of the cell expressing the DR5 proteins which were bound. Apoptosis will occur, as presently understood, when multiple receptor proteins are bound together, causing cross-linking of the receptor molecules such that a signal is transmitted across the cell membrane into the cytosol of the cell expressing DR5.

A dimeric, pentameric, or hexameric binding molecule as provided herein can cross-link at least three DR5 monomers expressed on the surface of a cell. Due to the dimeric, pentameric, or hexameric nature of a DR5 binding molecule as provided herein, the molecule can cross-link as many as three, four, five, six, seven, eight, nine, ten, eleven, or twelve DR5 monomers on a cell. The receptor proteins are then spatially brought into proximity of each other, thereby contributing to their cross-linking and activation. When all five or all six of the bivalent binding units a DR5 binding molecule as provided herein bind to a receptor, binding up to ten or twelve DR5 monomers on a single cell, respectively, cross-linking and activation of the receptors can occur.

Because each of the binding units is bivalent, each binding molecule can bind to as many as 10 (for pentameric binding molecules) or 12 (for hexameric binding molecules) DR5 monomers.

Upon activation of the receptors by the binding of a dimeric, pentameric, or hexameric binding molecule as provided herein, the cell can either undergo apoptosis as described above.

In certain aspects, a dimeric, pentameric, or hexameric binding molecule as presently disclosed can induce DR5-mediated apoptosis in a DR5-expressing cell at a higher potency than an equivalent amount of a bivalent IgG antibody or fragment thereof, which also specifically binds to and agonizes DR5. Not wishing to be bound by theory, because a provided binding molecule is dimeric, pentameric, or hexameric, and because each binding unit is bivalent, such a binding molecule can induce receptor-mediated functions previously characterized for DR5 at a higher potency than any single binding unit alone, such as an equivalent IgG binding unit. IgG binding units are bivalent, containing two binding sites, but as previous clinical studies have shown, binding of two DR5 receptors with a single IgG molecule can be ineffective without addition of other components, such as cross-linkers, etc.

By “potency” or “improved binding characteristics” is meant the least amount of a given binding molecule necessary to achieve a given biological result, e.g., activation of 20%, 50%, or 90% of DR5 monomers in a given assay, e.g., an ELISA or Western blot-based caspase assays, annexin-v staining as seen by FACS analysis, or other assay. Or a reduced tumor growth rate or increased survival in an in vivo tumor assay.

Because a binding molecule as provided herein is dimeric, pentameric, or hexameric, it can contain as many as 4, 10, or 12, respectively, antigen-binding domains. Each of the antigen-binding domains can specifically bind to and agonize DR5. Further, each antigen-binding domain can be specific for one particular epitope of DR5.

Thus, a single dimeric, pentameric, or hexameric binding molecule can: a) simultaneously bind a single epitope on DR5, or b) bind many different epitopes on DR5.

The binding units of a dimeric, pentameric, or hexameric binding molecule as provided herein can be human, humanized, or chimeric immunoglobulin binding units. Methods of humanizing immunoglobulin sequences are well known in the art. Thus, the nucleotide sequences encoding a dimeric, pentameric, or hexameric binding molecule polypeptide can be directly from human sequences, or can be humanized or chimeric, i.e., encoded by sequences from multiple different species.

The cells which express DR5 can be any animal cell. For instance, in one embodiment, the cell is a human cell. For example, the cell can be any one or more of primate, rodent, canine, equine, etc., cells. Further, the cell expressing DR5 can be a cancer cell. That is, the cell can be a cell in a tumor which is malignant or benign.

A dimeric, pentameric, or hexameric binding molecule as provided herein can be genetically engineered such that its antigen-binding domains are encoded by sequences known to specifically bind DR5. Many groups have published sequences of variable regions of monoclonal antibodies, most of the IgG isotype that are characterized and are known to specifically bind to DR5. Non-limiting immunoglobulin variable domain sequences that are known to specifically bind to DR5 are provided in Tables 2 and 3. One of skill in the art is capable of engineering these published sequences into immunoglobulin structures, such as an IgG, IgA, IgM structure, or biologically active or functional multimeric fragments variants, or derivatives thereof. Methods for genetically engineering cloned variable regions into immunoglobulin domains, and expressing and purifying such constructs are published and within the capability of one skilled in the art.

Thus, in certain aspects, a DR5 binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv amino acid sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

TABLE 2
Anti-DR5 Antibody VH (or Heavy Chain) and VL (or Light Chain) Sequences

| SEQ | | SEQ | | |
|---|---|---|---|---|
| ID | VH or Heavy Chain | ID | VL or Light Chain | Reference |
| 1 | EVQLVQSGGGVERPGGSLRLSCAASGFIFDDYGMS | 2 | SSELTQDPAVSVALGQTVRITCQGDSLRSYYAS | U.S. Pat. App. |
| | WVRQAPGKGLEWVSGINWNGGSTGYADSVKGRV | | WYQQKPGQAPVLVIYGKNNRPSGIPDRFSGSSSG | Pub. No. |
| | TISRDNAKNSLYLQMNSLRAEDTAVYYCAKILGAG | | NTASLTITGAQAEDEADYYCNSRDSSGNHVVFG | 20060269555A1 |
| | RGWYFDLWGKGTTVTVSS | | GGTKLTVL | |
| 3 | EVQLVQSGGGVERPGGSLRLSCAASGFIFDDYAMS | 4 | SELTQDPAVSVALGQTVRITCSGDSLRSYYASW | U.S. Pat. No. |
| | WVRQAPGKGLEWVSGINWQGGSTGYADSVKGRV | | YQQKPGQAPVLVIYGANNRPSGIPDRFSGSSSGN | 8,029,783 |
| | TISRDNAKNSLYLQMNSLRAEDTAVYYCAKILGAG | | TASLTITGAQAEDEADYYCNSADSSGNHVVFGG | |
| | RGWYFDYWGKGTTVTVSS | | GTKLTVL | |
| 5 | QVQLQESGPGLVKPSQTLSLTCTVSGGSISSGDYFW | 6 | EIVLTQSPGTLSLSPGERATLSCRASQGISRSYLA | U.S. Pat. No. |
| | SWIRQLPGKGLECIGHIHNSGTTYYNPSLKSRVTISV | | WYQQKPGQAPSLLIYGASSRATGIPDRFSGSGSG | 7,521,048 |
| | DTSKKQFSLRLSSVTAADTAVYYCARDRGGDYYY | | TDFTLTISRLEPEDFAVYYCQQFGSSPWTFGQGT | |
| | GMDVWGQGTTVTVSS | | KVEIK | |
| 7 | EVQLVESGGGLVQPGGSLRLSCAASGFTFSSYVMS | 8 | DIQMTQSPSSLSASVGDRVTITCKASQDVGTAVA | U.S. Pat. No. |
| | WVRQAPGKGLEWVATISSGGSYTYYPDSVKGRFTI | | WYQQKPGKAPKLLIYWASTRHTGVPSRFSGSGS | 7,790,165 |
| | SRDNAKNTLYLQMNSLRAEDTAVYYCARRGDSMI | | GTDFTLTISSLQPEDFATYYCQQYSSYRTFGQGT | |
| | TTDYWGQGTLVTVSSASTKGPSVFPLAPSSKSTSGG | | KVEIKRTVAAPSVFIFPPSDEQLKSGTASVVCLLN | |
| | TAALGCLVKDYFPEPVTVSWNSGALTSGVHTFPAV | | NFYPREAKVQWKVDNALQSGNSQESVTEQDSK | |
| | LQSSGLYSLSSVVTVPSSSLGTQTYICNVNHKPSNT | | DSTYSLSSTLTLSKADYEKHKVYACEVTHQGLS | |
| | KVDKRVEPKSCDKTHTCPPCPAPELLGGPSVFLFPP | | SPVTKSFNRGEC | |
| | KPKDTLMISRTPEVTCVVVDVSHEDPEVKFNWYVD | | | |
| | GVEVHNAKTKPREEQYNSTYRVVSVLTVLHQDWL | | | |
| | NGKEYKCKVSNKALPAPIEKTISKAKGQPREPQVYT | | | |
| | LPPSREEMTKNQVSLTCLVKGFYPSDIAVEWESNG | | | |
| | QPENNYKTTPPVLDSDGSFFLYSKLTVDKSRWQQG | | | |
| | NVFSCSVMHEALHNHYTQKSLSLSPGK | | | |
| 9 | QIQLVQSGPE LKKPGETVKI SCKASGYTFT | 10 | DVVMTQTPLS LPVSLGDQAS ISCRSSQSLV | U.S. Pat. No. |
| | DFSMNWVKQA PGKGLKWMGW INTETGEPTY | | HSNGNTYLHW YLQKPGQSPK LLIYKVSNRF | 7,893,216 |
| | ADDFKGRFAL SMETSASTAY LQINNLKNED | | SGVPDRFSGS GSGTDFTLKI SRVEAEDLGV | |
| | TATYFCVRID YWGQGTTLTV SS | | YFCFQSTHVP HTFGGGTKLE IKR | |
| 11 | MDWTWRILFLVAAATSAHSQVQLVQSGAEMKKPG | 12 | MEAPAQLLFLLLLWLPDTTGEIVLTQSPATLSLS | U.S. Pat. No. |
| | ASVKVSCKTSGYTFTNYKINWVRQAPGQGLEWMG | | PGERATLSCRASQSVSSYLAWYQQKPGQAPRLL | 7,115,717 |
| | WMNPDTDSTGYPQKFQGRVTMTRNTSISTAYMELS | | IYDASNRATGIPARFSGSGSGTDFTLTISSLEPEDF | |
| | SLRSEDTAVYYCARSYGSGSYYRDYYYGMDVWG | | AVYYCQQRSNWPLTFGGGTKVEIKR | |
| | QGTTVTVSS | | | |
| 13 | MKHLWFFLLL VAAPRWVLSE VQLQQSGPEL | 14 | MVLQTQVFIS LLLWISGAYG DVVMTQTPLS | EP Pat. |
| | VKPGASVKIS CKASGYSFIG YFMNWMKQSH | | LPVSLGDQAS ISCRSSQSLV HSNGNTYLHW | Publication No. |
| | GKSLEWIGRF NPYNGDTFYN QKFKGKATLT | | YLQKPGQSPK LLIYKVSNRF SGVPDRFSGS | EP2636736A1 |
| | VDKSSTTAHM ELLSLTSEDS AVYFCGRSAY | | GSGTDFTLKI SRVEAEDLGI YFCSQSTHVP | |
| | YFDSGGYFDY WGQGTTLTVS SASTKGPSVF | | WTFGGGTKLE IKRTVAAPSV FIFPPSDEQL | |
| | PLAPSSKSTS GGTAALGCLV KDYFPEPVTV | | KSGTASVVCL LNNFYPREAK VQWKVDNALQ | |
| | SWNSGALTSG VHTFPAVLQS SGLYSLSSVV | | SGNSQESVTE QDSKDSTYSL SSTLTLSKAD | |
| | TVPSSSLGTQ TYICNVNHKP SNTKVDKRVE | | YEKHKVYACE VTHQGLSSPV TKSFNRGEC | |
| | PKSCDKTHTC PPCPAPELLG GPSVFLFPPK | | | |
| | PKDTLMISRT PEVTCVVVDV SHEDPEVKFN | | | |
| | WYVDGVEVHN AKTKPREEQY NSTYRVVSVL | | | |
| | TVLHQDWLNG KEYKCKVSNK ALPAPIEKTI | | | |
| | SKAKGQPREP QVYTLPPSRE EMTKNQVSLT | | | |
| | CLVKGFYPSD IAVEWESNGQ PENNYKTTPP | | | |
| | VLDSDGSFFL YSKLTVDKSR WQQGNVFSCS | | | |
| | VMHEALHNHY TQKSLSLSPG K | | | |
| 15 | QVQLVQSGSELKKPGASVKVSCKASGYTFTDFSMN | 16 | DIVMTQSPLSLPVTPGEPASISCRSSQSLVHSNGN | PCT Publication |
| | WVRQAPGQGLEWMGWINTETGEPTYADDFKGRFV | | TYLHWYLQKPGQSPQLLIYKVSNRFSGVPDRFS | No. WO |
| | FSLDTSVSTAYLQISSLKAEDTAVYYCARIDYWGQ | | GSGSGTDFTLKISRVEAEDVGVYYCFQSTHVPHT | 2014/063368 Al |
| | GTTVTVSS | | FGQGTKLEIKR | |
| 17 | MGXLGLSWVFLVVILEGVQCEVHLVESGGGLVRPG | 18 | MRLPAQLLGLLMLWVSGSSGDIQMTQSSSSFSV | U.S. Pat. No. |
| | GSLKLSCAASGFAFSSYDMSWVRQTPEKRLEWVA | | SLGDRVTITCKASEDIYNRLAWYQQKPGNAPRL | 7,897,730 |
| | YISDGGGITYYPDTMKGRFTISRDNAKNTLSLQMSS | | LISGATSLETGVPSRFSGSGSGKDYTLSITSLQTE | |
| | LKSEDTAMYYCARHITMVVGPFAYWGQGTLVTVS | | DVATYYCQQMSTPLTFGAGTKLELKRAVAAP | |
| | AASTKGPSVFPLAPCSRSTSGGTAALGCLVKDYFPE | | SVDIFPPSDEQLKSGTASVVCLLNNFYPREAKVQ | |
| | PVTVSWNSGALTSGVHTFPAVLQSSGLYSLSSVVTV | | WKVDNALQSGNSQESVTEQDSKDSTYSLSSTLT | |
| | PSSSLGTQTYICNVNHKPSNTKVDKRVEPKSCDKTH | | LSKADYEKHKVYACEVTHQGLSSPVTKSFNRGE | |
| | TCPPCPAPELLGGPSVFLFPPKPKDTLMISRTPEVTC | | C | |
| | VVVDVSHEDPEVKFNWYVDGVEVHNAKTKPREEQ | | | |
| | YNSTYRVVSVLTVLHQDWLNGKEYKCKVSNKGLP | | | |
| | APIEKTISKAKGQPREPQVYTLPPSREEMTKNQVSL | | | |
| | TCLVKGFYPSDIAVEWESNGQPENNYKTTPPVLDS | | | |
| | DGSFFLYSKLTMDKSRWQQGNVFSCSVMHEALHN | | | |
| | HYTQKSLSLSPGK | | | |
| 19 | MELGLSWVFLVVILEGVQCEVQLQQSGPELVKPGA | 20 | MRLPAQLLGLLMLWVSGSSGDIVMTQSHKFMS | U.S. Pat. No. |
| | SVRMSCKASGYTFTSYFIHWVKQRPGQGLEWIGWI | | TSVGDRVSITCKASQDVSTAVAWYQQKPGQSPR | 7,897,730 |
| | YPGNVNTKYSEKFKGKATLTADKSSSTAYMQFSSL | | LLIYWASTRHTGVPDRFTGSGSGTDYTLTISSVQ | |
| | TSEDSAVYFCARGEAGYFDYWGQGTTLTVSSASTK | | AEDQALYYCQQHYRTPWTFGGGTKLEIKRAVA | |
| | GPSVFPLAPCSRSTSGGTAALGCLVKDYFPEPVTVS | | APSVDIFPPSDEQLKSGTASVVCLLNNFYPREAK | |
| | WNSGALTSGVHTFPAVLQSSGLYSLSSVVTVPSSSL | | VQWKVDNALQSGNSQESVTEQDSKDSTYSLSST | |
| | GTQTYICNVNHKPSNTKVDKRVEPKSCDKTHTCPP | | LTLSKADYEKHKVYACEVTHQGLSSPVTKSFNR | |
| | CPAPELLGGPSVFLFPPKPKDTLMISRTPEVTCVVVD | | GEC | |
| | VSHEDPEVKFNWYVDGVEVHNAKTKPREEQYNST | | | |
| | YRVVSVLTVLHQDWLNGKEYKCKVSNKGLPAPIE | | | |
| | KTISKAKGQPREPQVYTLPPSREEMTKNQVSLTCLV | | | |
| | KGFYPSDIAVEWESNGQPENNYKTTPPVLDSDGSFF | | | |
| | LYSKLTMDKSRWQQGNVFSCSVMHEALHNHYTQK | | | |
| | SLSLSPGK | | | |
| 21 | QVQLVQSGAEVKKPGASVKVSCKASGYTFTSYDIN | 22 | DIQMTQSPSSLSASVGDRVTITCRASQSISIYLNW | U.S. Pat. No. |
| | WVRQATGQGLEWMGWMNPNSDNTGYAQKFQGR | | YQQKPGKAPKLLIYAASSLQSGVPLRFSGSGSGT | 7,521,048 |
| | VTMTRNTSISTAYMELSSLRSEDTAVYYCARWNHY | | DFTLTISSLQPEDIATYYCQQSYKTPLTFGGGTK | |
| | GSGSHFDYWGQGTLVTVSS | | VEIK | |
| 23 | QVQLQESGPGLVKPSQTLSLTCTVSGGSISSGGHYW | 24 | DIQMTQSPSSLSASVGDRVTITCRASQGLRNDLG | U.S. Pat. No. |
| | SWIRQHPGKGLEWIGYIYYSGSTYYNPSLKSRVTIS | | WFQQKPGKVTKRLIYAASSLQRGVPSRFSGSGS | 7,521,048 |
| | VDTSKNQFSLKLSSVTAADTAVYYCARDDSSGWGF | | GTEFTLTISSLQPEDFATYYCLQHYSFPWTFGQG | |
| | DYWGQGILVTVSS | | TKVEIK | |
| 25 | QVQLQESGPGLVKPSQTLSLTCTVSGGSISSGGHYW | 26 | DIQMTQSPSSLSASVGDRVTITCRASQGLRNDLG | U.S. Pat. No. |
| | SWIRQHPGKGLEWIGYIYYSGSAYYNPSLKSRVTIS | | WFQQKPGKAPKRLIYAASSLQRGVPSRFSGSGS | 7,521,048 |
| | VDTSKNQFSLKLSSVTAADTAVYYCARDDSSGWGF | | GTEFTLTISSLQPEDFTTYFCLQHNSFPWTFGQGT | |
| | DYWGQGILVTVSS | | KVEIK | |
| 27 | QVQLQESGPGLVKPSQTLSLTCTVSGGSISSGGHYW | 28 | DIQMTQSPSSLSASVGDRVTITCRASQGLRNDLG | U.S. Pat. No. |
| | SWIRQHPGKGLEWIGYIYYSGSAYYNPSLKSRVTIS | | WFQQKPGKAPKRLIYAASSLQRGVPSRFSGSGS | 7,521,048 |
| | VDTSKNQFSLKLSSVTAADTAVYYCARDDSSGWGF | | GTEFTLTISSLQPEDFTTYFCLQHNSFPWTFGQGT | |
| | DYWGQGILVTVSS | | KVEIK | |
| 29 | QVQLVESGGGLVKPGGSLRLSCAASGFTFSDYYMN | 30 | DIQMTQSPSSLSASVGDRVTITCRSSQSISNYINW | U.S. Pat. No. |
| | WIRQAPGKGLEWVSHISSSGSILDYADSVKGRFTISR | | YQQRPGKAPNLLIHDVSSFQSAVPSRFSRSGSGT | 7,521,048 |
| | DNAKNSLYLQMNSLRVEDTAVYYCARDGAAAGT | | VFTLTISSLQPEDFATYFCQQTYITPFTFGPGTKV | |
| | DAFDLWGQGTMVTVSS | | DIK | |
| 31 | QVQLVESGGGVVQPGRSLRLSCAASGFTFSYYGIH | 32 | DIQMTQSPSSLSASVGDRVTITCRASQGISNYLA | U.S. Pat. No. |
| | WVRQAPGKGLEWVAVIWYDGSNKYYADSVKGRF | | WYQQKPGKVPKLLIYAASTLQSGVPSRFSGSGS | 7,521,048 |
| | TISRDNSKNTLYLQMNSLRAEDTAVYYCARGRYSS | | GTDFTLTISSLQPEDVATYYCQKYNSAPLTFGGG | |
| | SSWWYFDLWGRGTLVTVSS | | TKVEIK | |
| 33 | QVQAEQSGPGLVKPSETLSLTCTVSGGSISNYYWS | 34 | DIVMTQSPDSLAVSLGERATINCKSSQSVLYRSN | U.S. Pat. No. |
| | WIRQPPGKGLEWIGYIYYSGSTKYNPSLKSRVTISV | | NKIYLAWYQQKPGQPPKLLIYWASTRESGVPDR | 7,521,048 |
| | DTSKNQFSLKLTSVTTADTAVYYCARDSPRGFSGY | | FSGSGSGTDFTLTISSLLAEDVAVYYCQQYYSTP | |
| | EAFDSWGQGTLVTVSS | | FTFGPGTKVDIK | |
| 35 | QVQLQESGPGLVKPSQTLSLTCTVSGGSISSDNYYW | 36 | DIVMTQSPLSLPVTPGEPASISCRSSQSLLRRNGY | U.S. Pat. No. |
| | SWIRQHPGKGLEWIGYIYYSGSTYYNPSLKSRVTIS | | NYLDWYLQKPGQSPQLLIYLGSNRASGVPDRFS | 7,521,048 |
| | VDTSKNQFSLKLSSVTAADTAVYYCARGVNWNFL | | GSGSGTDFTLKISRVEAEDVGVYYCMQALQTPL | |
| | FDIWGQGTMVTVSS | | TFGGGTEVEIK | |
| 37 | QVQLVESGGGLVKPGGSLRLSCAASGFTFSDYYMS | 38 | DIVMTQFPDSLAVSLGERATINCKSSQSVLHSSN | U.S. Pat. No. |
| | WIRQAPGKGLEWVSYISRSGSTIYYADSVKGRFTIS | | NKNYLTWYQLKPGQPPKLLIYWASTRESGVPDR | 7,521,048 |
| | RDNAKNSLYLQMNSLRAEDTAVYYCARSLGGMDV | | FSGSGSGTDFTLTISSLQAEDVAVYYCHQYYSTP | |
| | WGQGTTVTVSS | | SSFGQGTKLEIK | |
| 39 | QVQLVESGGGVVQPGRSLRLSCAASGFTFNNYGM | 40 | DIQMTQSPSSLSASVGDRVTITCRTSQSISTYLNW | U.S. Pat. No. |
| | HWVRQAPGKGLEWVAVIWYDGSNKYYADSVKGR | | YQQKPGKAPKLLISATSSLQSGVPSRFSGSGSGT | 7,521,048 |
| | FTISRDNSKNTLYLQMNSLRAEDTAVYYCARDRTV | | DFTLTISSLQPEDFATYYCQQSYSTPLTFGGGTK | |
| | YSNSSPFYYYYYGMDVWGQGTTVTVSS | | VEIK | |
| 41 | QVQLVESGGGVVQPGRSLRLSCAASGFTFSTYGMH | 42 | DIQMTQSPSSLSASVGDRVTITCRASQSISSYLNW | U.S. Pat. No. |
| | WVRQAPGKGLEWVAVIWYDGSNKYYADSVKGRF | | YQQKPGKAPKLLISATSSFQSGVPSRFSGSGSGT | 7,521,048 |
| | TISRDNSKNTLYLQMNSLRAEDTAVYYCARDRTVY | | DFTLTISSLQPEDFAAYYCQQSYSTPLTFGGGTK | |
| | SSSSPFYYYYYGMDVWGQGTTVTVSS | | VEIK | |
| 43 | QVQLQQWGARLLKPSETLSLTCAVYGGSFSGYYW | 44 | DIVMTQSPDSLAVSLGERATINCKSSQSVLHSSN | U.S. Pat. No. |
| | SWIRQPPGKGLEWIGEINHSGSTNYNPSLKSRVTISV | | NKNYLVWYQQKPGQPPKLLIYWASTRESGVPD | 7,521,048 |
| | DTSKNQFSLKLRSVTAADTAVYYCARGGSSGYWY | | RFSGSGSGTDFTLTISSLQAEDVAVYYCQQYYST | |
| | FDLWGRGTLVTVSS | | PLTFGGGTKVEIK | |
| 45 | EVQVVESGGGLVKPGGSLRLSCAASGFTFSSYSMN | 46 | DIQMTQSPSSVSASVGDRVTITCRASQGISSWLV | U.S. Pat. No. |
| | WVRQAPGKGLEWVSSISSSSSYIYYADSVKGRFTIS | | WYQQKPGKAPKLLIYAASSLQSGVPSRFSGSGS | 7,521,048 |
| | RDNAKNSLYLQMNSLRAEDTAVYYCARGGSSWYG | | GTDFTLTISSLQPEDFATYYCQQANSFPFTFGGG | |
| | DWFDPWGQGTLVTVSS | | TKVEIK | |
| 47 | QLVESGGGVVQPGRSLRLSCAASGFTFSSYGMHWV | 48 | DIQMTQSPSSLSASVGDRVTITCRASQGISNYLA | U.S. Pat. No. |
| | RQAPGKGLEWVAVIWYDGRNKYYADSVKGRFTIS | | WFQQKPGKAPKSLIYAASSLQSGVPSKFSGSGSG | 7,521,048 |
| | RDNSKNTLYLQMNSLRAEDTAVYYCAREVGYCTN | | TDFTLTISSLQPEDFATYYCQQYNSYPLTFGGGT | |
| | GVCSYYYYGMDVWGQGTTVTVSS | | KVEIK | |
| 49 | QVQLQESGPGLVKPSQTLSLTCSVSGGSISSGGYYW | 50 | DIQMTQSPSSVSASVGDRVTITCRASQGISSWLA | U.S. Pat. No. |
| | SWIRQHPGKGLEWIGYIYYSGSTYCNPSLKSRVTIS | | WYQQKPGKAPKFLIFVASSFQSGVPSRFSGSGSG | 7,521,048 |
| | VDTSKNQFSLKLSSVTAADTAVYYCARDNGSGSYD | | TDFTLTISSLQPEDFATYYCQQANSFPRTFGQGT | |
| | WFDPWGQGILVTVSS | | KVEIK | |
| 51 | QVQMQESGPGLVKPSQTLSLTCTVSGGSISSGDYY | 52 | DIQMTQSPSSVSASVGDRVTITCRASQGISSWLA | U.S. Pat. No. |
| | WSWIRQHPGKNLEWIGYIYYSGSTYYNPSLKSRVTI | | WYQQKPGKAPKFLIFVASSLQSGVPSRFSGSGSG | 7,521,048 |
| | SVDTSKNQFSLKLSSVTAADTAVYYCARDNGSGSY | | TDFTLTISSLQPEDFATYYCQQANSFPRTFGQGT | |
| | DWFDPWGQGTLVTVSS | | KVEIK | |
| 53 | KVQLQQSGAELVKPGASVKLSCKASGYTFTDYTIH | 54 | DIAMTQSHKFMSTLVGDRVSITCKASQDVNTAI | U.S. Pat. No. |
| | WVKQRSGQGLEWIGWFYPGGGYIKYNEKFKDRAT | | AWYQQKPGQSPKLLIYWASTRHTGVPDRFTGSG | 7,229,617 |
| | LTADKSSNTVYMELSRLTSEGSAVYFCARHEEGIYF | | SGTDYTLTISSMEAEDAATYYCQQWSSNPLTFG | |
| | DYWGQGTTLTVSS | | AGTKLELKRA | |
| 55 | KVQLQQSGAELVKPGASVKLSCKASGYTFTDYTIH | 56 | DIVMTQSHKFMSTSVGDRVSITCKASQDVNTAI | U.S. Pat. No. |
| | WVKQRSGQGLEWIGWFYPGGGYIKYNEKFKDRAT | | AWYQQKPGQSPKLLIYWASTRHTGVPDRFTGSG | 7,229,618 |
| | LTADKSSNTVYMELSRLTSEDSAVYFCARHEEGIYF | | SGTDYTLTISSVQAEDLALYYCQQHYTTPFTFGS | |
| | DYWGQGTTLTVSS | | GTKL | |
| 82 | MDLMCKKMKHLWFFLLLVAAPRWVLSQLQLQES | 83 | MEAPAQLLFLLLLWLPDTTGEIVLTQSPATLSLS | U.S. Pat. No. |
| | GPGLVKPSETLSLTCTVSGGSIISKSSYWGWIRQPPG | | PGERATLSCRASQSVSSFLAWYQQKPGQAPRLLI | 7,115,717 |
| | KGLEWIGSIYYSGSTFYNPSLKSRVTISVDTSKNQFS | | YDASNRATGIPARFSGSGSGTDFTLTISSLEPEDF | |
| | LKLSSVTAADTAVYYCARLTVAEFDYWGQGTLVT | | AVYYCQQRSNWPLTFGPGTKVDIKRT | |
| | VSSAS | | | |
| 84 | MDLMCKKMKHLWFFLLLVAAPRWVLSQLQLQES | 85 | MEAPAQLLFLLLLWLPDTTGEIVLTQSPATLSLS | U.S. Pat. No. |
| | GPGLVKPSETLSLTCTVSGGSISSRSNYWGWIRQPP | | PGERATLSCRASQSVSSFLAWYQQKPGQAPRLLI | 7,115,717 |
| | GKGLEWIGNVYYRGSTYYNSSLKSRVTISVDTSKN | | YDASNRATGSPARFSGSGSGTDFTLTISSLEPEDF | |
| | QFSLKLSSVTVADTAVYYCARLSVAEFDYWGQGIL | | AVYYCQQRSDWPLTFGPGTKVDIKRT | |
| | VTVSSAS | | | |
| 86 | MDLMCKKMKHLWFFLLLVAAPRWVLSQLQLQES | 87 | METPAQLLFLLLLWLPDTTGEIVLTQSPGTLSLSP | U.S. Pat. No. |
| | GPGLVKPSETLSLTCTVSGGSISSSSYYWGWVRQPP | | GERATLSCRASQSVSSSYLAWYQQKPGQAPRLL | 7,115,717 |
| | GKGLEWIGSIHYSGSTFYNPSLKSRVTISVDTSKNQF | | IYGASSRATGIPDRFSGSGSGTDFTLTISRLEPEDF | |
| | SLKLSSVTAADTTVYYCARQGSTVVRGVYYYGMD | | AVYYCQQYGSSPLYTFGQGTKLEIKRT | |
| | VWGQGTTVTVSSAS | | | |
| 88 | MEFGLSWLFLVAILKGVQCEVQLLESGGGLVQPGR | 89 | MSPSQLIGFLLLWVPASRGEIVLTQSPDFQSVTPK | U.S. Pat. No. |
| | SLRLSCAASGFTFSSYAMSWVRQAPGKGLEWVSAI | | EKVTITCRASQSIGSSLHWYQQKPDQSPKLLIKY | 7,115,717 |
| | SGSGGSRYYADSVKGRFTISRDNSKNTLYLQMNSL | | ASQSFSGVPSRFSGSGSGTDFTLTINSLEAEDAAA | |
| | RAEDTAVYYCAKESSGWFGAFDYWGQGTLVTVSS | | YYCHQSSSLPITFGQGTRLEIKR | |

TABLE 3
Anti DR5 ScFv Sequences
SEQ

| ID | SEQUENCE | Reference |
|---|---|---|
| 57 | EVQLVQSGGGVERPGGSLRLSCAASGFTFDDYGMSWVRQAPGKGLEWV | U.S. Pat. |
| | SGINWNGGSTGYADSVKGRVTISRDNAKNSLYLQMNSLRAEDTAVYYCA | Application |
| | KILGAGRGWYFDLWGKGTTVTVSSGGGGSGGGGSGGGGSSELTQDPAVS | Publication |
| | VALGQTVRITCQGSLRSYYASWYQQKPGQAPVLVIYGKNNRPSGIPDRF | No. |
| | SGSSSGNTASLTITGAQAEDEADYYCNSRDSSGNHVVFGGGTKLTVLG | 2006/0269555 |
| 58 | EVQLVETGGGLVQPGGSLRLSCAASGFTFSSYAMSWVRQAPGKGLEWVS | U.S. Pat. |
| | AISGSGGSTYYADSVKGRFTISRDNSKNTLYLQMNSLRAEDTAVYHCARG | Application |
| | GYSSSRSAAYDIWGQGTLVTVSSGGGGSGGGGSGGGGSSELTQDPAVSV | Publication |
| | ALGQTVRITCQGDSLRSYYASWYQQKPGQAPVLVIYGKNNRPSGIPDRFS | No. |
| | GSSSGNTASLTITGAQAEDEADYYCNSRDSSGNHVVFGGGTKLTVLG | 2006/0269556 |
| 59 | QVQLVQSGAEVKKPGASVKISCEGSGYTFNSYTLHWLRQAPGQRLEWM | U.S. Pat. |
| | GRINAGNGNTKYSQNFQGRLSITRDTSATTAYMELSSLRSEDTGVYYCAR | Application |
| | VFTYSFGMDVWGRGTLVTVSSGGGGSGGGGSGGGGSAQSVLTQPPSASG | Publication |
| | TPGQRVTISCSGGGSNIGRNSVSWYQQLPGTAPKLILYSNNQRPSGVPDRF | No. |
| | SGSKSGTSASLAISGLRSEDEALYYCAAWDDSLSGGVFGGGTKLTVLG | 2006/0269557 |
| 60 | QVQLVESGGGLVQPGGSLRLSCAASGFTFSSYAMSWVRQAPGKGLEWVS | U.S. Pat. |
| | AISGSGGSTYYADSVKGRFTISRDNSKNTLYLQMNSLRAEDTAVYYCAK | Application |
| | VHRPGRSGYFDYWGRGTLVTVSSGGGGSGGGGSGGGGSSELTQDPAVSV | Publication |
| | ALGQTVRITCQGDSLRSYYASWYQQKPGQAPVLVIYGKNNRPSGIPDRFS | No. |
| | GSSSGNTASLTITGAQAEDEADYYCNSRDSSGNHVVFGGGTKLTVLG | 2006/0269558 |
| 61 | QVQLQQSGAEVKKPGASVRVSCQASGYSLSEYYIHWVRQAPGQGLEWM | U.S. Pat. |
| | GWLNPNSGVTDYAQKFQGRVSMTRDTSISTAYMELSSLTFNDTAVYFCA | Application |
| | RGNGDYWGKGTLVTVSPGGGGSGGGGSGGGGSSELTQDPAVSVALGQT | Publication |
| | VRITCQGDSLRSYYTNWFQQKPGQAPLLVVYAKNKRPSGIPDRFSGSSSG | No. |
| | NTASLTITGAQAEDEADYYCHSRDSSGWVFGGGTKLTVLG | 2006/0269559 |
| 62 | QVQLVQSGGGVVQPGRSLRLSCAASGFTFSPDAMHWVRQAPGKGLEWM | U.S. Pat. |
| | GVISFDGSQTFYADSVKGRFTISRDNSQNTLYLQMNSLRSDDTAVYYCAR | Application |
| | APARFFPLHFDIWGRGTMVTVSSGGGGSGGGGSGGGGSALSSELTQDPA | Publication |
| | VSVALGQTVRITCQGDSLRTHYASWYHQRPGRAPVLVNYPKDSRPSGIPD | No. |
| | RFSGSSSGNTASLTITGAQAADEGDYYCQSRDSSGVLFGGGTKVTVLG | 2006/0269560 |
| 63 | EVQLVESGGGLVQPGGSLRLSCAASGFTFSSYWMSWVRQAPGKGLEWV | U.S. Pat. |
| | ANIKQDGSEKYYVDSVKGRFTISRDNAKNSLYLQMNSLRAEDTAVYYCA | Application |
| | RDFSGYGDYLDYWGKGTLVTVSSGGGGSGGGGSGGGGSAQSALTQPPS | Publication |
| | ASGSPGQSVTISCTGTSSDIGNYNYVSWYQQHPGKAPKLMIYEVNERPSG | No. |
| | VPDRFSGSKSGNTASLTVSGLRPEDEADYYCSSYAGNNAVIFGGGTQLTV | 2006/0269561 |
| | LG | |
| 64 | QVQLVQSGAEVKKPGASVKVSCKASGYTFTTHAMHWVRQAPGQSLEW | U.S. Pat. |
| | MGWINTGNGNTKYSQSFQGRVSITRDTSANTAYMELSSLKSEDTAMYYC | Application |
| | ARASRDSSGYYYVPPGDFFDIWGQGTLVTVSSGGGGSGGGGSGGGGSAQ | Publication |
| | SALTQPASVSGSPGQSITISCTGSRSDIGGYNFVSWYQQHPGKAPKLLIYD | No. |
| | VYNRPSGISDHFSGSKSDNTASLTISGLQSEDDADYYCSSYAGYHTWIFGG | 2006/0269562 |
| | GTKVTVLG | |
| 65 | EVQLVQSGAEVKKPGASVKLSCKASGYTLVNYFMHWVRQAPGQGPEW | U.S. Pat. |
| | MGMINPSGGTTKNRQKFQDRVTMTRDTSTRTVYMELSGLTSEDTAVYYC | Application |
| | ATDFKGTDILFRDWGRGTLVTVSSGGGGSGGGGSGGGGSAQSVLTQPPS | Publication |
| | ASGTPGQRVSISCSGSSSNIGSNTVIWYQQLPGTAPKLLMYSNDRRPSGVP | No. |
| | DRFSGSKSGTSASLAISGLQSEDEADYYCATWDDSLNGHYVFGTGTKLTV | 2006/0269563 |
| | LG | |
| 66 | QMQLVQSGGGLVKPGGSLRLSCAASGFTFSDYYMSWIRQAPGKGLEWVS | U.S. Pat. |
| | AISGSGGSTYYADSVKGRFTISRDNSKNTLYLQMNSLRAEDTAVYYCARG | Application |
| | GSTFDIWGRGTMVTVSSGGGGSGGGGSGGGGSAQPVLTQPPSASGTPGQ | Publication |
| | RVTISCSGSNSNIGSRPVNWYQQLPGTAPKLLIQGNNQRPSGVPDRFSGSK | No. |
| | SGTSASLAISGLQSEDEADYYCAAWDDSLTGYVFGPGTKLTVLG | 2006/0269564 |
| 67 | QMQLVQSGGAVVQPGRSLRLSCAASGFTFSSYGMHWVRQAPGKGLEWV | U.S. Pat. |
| | AVISYDGSIKYYADSVKGRFTISRDNSKNTLYLQMNSLRAEDTAVYYCAR | Application |
| | ERLRGLDPWGQGTMVTVSSGGGGSGGGGSGGGGSSELTQDPAVSVALG | Publication |
| | QTVRITCQGDSLRSYYASWYQQKPGQAPVLVIYGKNNRPSGIPDRFSGSSS | No. |
| | GNTASLTITGAQAEDEADYYCNSRDSSGNHVVFGGGTKLTVLG | 2006/0269565 |
| 68 | EVQLVETGGGLVQPGGSLRLSCAASGFTFSPYYMSWVRQAPGKGLEWVS | U.S. Pat. |
| | AISGSGGSIYYADSVKGRFTISRDNSKNTLYLQMNSLRAEDTALYYCARG | Application |
| | ASGPDYWGRGTMVTVSSGGGGSGGGGSGGGGSAQSVLTQPPSVSAAPG | Publication |
| | QKVTISCSGSTSNIGNNYVSWYQQVPGTAPKLLIYDNNKRPSGIPDRFSGS | No. |
| | KSGTSATLGITGLQTGDEADYYCGTWDSSLSALVFGGGTKVTVLG | 2006/0269566 |
| 69 | QVQLQQSGAEVKTPGSSVKVSCKASGGTFRNNAISWVRQAPGQGLEWM | U.S. Pat. |
| | GGFIPKFGTTNHAQKFQGRVTMTADDSTNTVYMELSSLRSEDTAVYYCA | Application |
| | RGGAYCGGGRCYLYGMDVWGQGTLVTVSSGGGGSGGGGSGGGGSAQA | Publication |
| | VVIQEPSLTVSPGGTVTLTCGSSTGAVTSGHYPYWFQQKPGQAPRTLIYDT | No. |
| | SNKRSWTPARFSGSLLGGKAALTLSGAQPEDEAEYYCLVSYSGSLVVFGG | 2006/0269567 |
| | GTKLTVLG | |
| 70 | EVQLLESGGGLVQPGGSLRLSCAASGFTFSSYAMSWVRQAPGKGLEWVS | U.S. Pat. |
| | AISGSGGSTYYADSVKGRFTISRDNSKNTLYLQMNSLRAEDTAVYYCVK | Application |
| | GAWLDYWGRGTMVTVSSGGGGSGGGGSGGGGSALNFMLTQPHSVSESP | Publication |
| | GKTVTISCTGSSGSVARNYVQWYQQRPGSAPTIVIYEDNRRPSGVPGRFSG | No. |
| | SIDRSSNSASLTISGLQTEDEADYYCQSYNYNTWVFGGGTKLTVLG | 2006/0269568 |
| 71 | EVQLVQSGAEVKKPGASVKVSCRASGYTFTSYGITWVRQAPGQGLEWM | U.S. Pat. |
| | GWISAYNGKTNYVQELQGRVTMTTDTSTSTVYMELTSLRSDDTAVYYCA | Application |
| | RRGNNYRFGYFDFWGQGTLVTVSSGGGGSGGGGSGGGGSALETTLTQSP | Publication |
| | GTLSLSPGERATLSCRASQSISSSNLAWYQQKPGRAPRLLIYGASSRAIGIP | No. |
| | DRFSGSGSGTDFTLTISRLEAEDFAVYYCQQYGSSPITFGQGTRLEIKR | 2006/0269569 |
| 72 | QVQLQQSGPG LVKPSQTLSL TCAISGDSVS STTVAWDWIR | U.S. Pat. |
| | QSPSRGLEWL GRTYYRSKWY NEYAVSVKSR ITINVDTSKN | No. 8,097,704 |
| | QISLQLNSVT PEDTAVYYCA REPDAGRGAF DIWGQGTTVT | |
| | SPLRWGRFGW RGLGRGWLRS PVTQSPGTLS LSPGERATLS | |
| | CRASQSVSSS HLAWYQQKPG QAPRLLIYGA SSRATGIPDR | |
| | FSGSGSGTDF TLTISSLEPE DFAVYYCQQR SNWPPRAVFG QGTRLEIK | |
| 73 | QVQLQQSGPG RVQPSQTLSL TCAISGDSVS NNNAAWYWIR | U.S. Pat. |
| | QSPSRGLEWL GRTYYRSKWY NDYAVSVKSR ITISPDTSKN | No. 8,097,705 |
| | QFSLQLNSVT PEDTAVYYCA RRGDGNSYFD YWGQGTLVTV | |
| | SSGILRWGRF GWRGLGRGWL EIVLTQSPGT LSLSPGERAT | |
| | LSCRASQSVS SGYVSWYRQK PGQAPRLLIY GASTRATGIP | |
| | DRFSGSGSGT DFTLTISRLE PEDFAVYYCH QYGSSPNTYG QGTKVGIK | |

In certain aspects the DR5 binding domain comprises a VH and a VL, wherein the VH and VL comprise amino acid sequences at least 60%, at least 65%, at least 70%, at least 75%, at least 80%, at least 85%, at least 90%, at least 95% or 100% identical to SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or where the VH and VL are situated in an ScFv comprising an amino acid sequence at least 60%, at least 65%, at least 70%, at least 75%, at least 80%, at least 85%, at least 90%, at least 95% or 100% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

While a variety of different dimeric, pentameric, and hexameric binding molecules can be contemplated by a person of ordinary skill in the art based on this disclosure, and as such are included in this disclosure, in certain aspects, a binding molecule as described above is provided in which each binding unit comprises two IgA or IgM heavy chains each comprising a VH situated amino terminal to the IgA or IgM constant region or fragment thereof, and two immunoglobulin light chains each comprising a VL situated amino terminal to an immunoglobulin light chain constant region.

Moreover, in certain aspects, at least one binding unit of the binding molecule, or at least two, at least three, at least four, at least five, or at least six binding units of the binding molecule, comprises or comprise two of the DR5 binding domains as described above. In certain aspects the two DR5 binding domains in the at least one binding unit of the binding molecule, or at least two, at least three, at least four, at least five, or at least six binding units of the binding molecule, can be different from each other, or they can be identical.

In certain aspects, the two IgA or IgM heavy chains within the at least one binding unit of the binding molecule, or at least two, at least three, at least four, at least five, or at least six binding units of the binding molecule, are identical. In certain aspects, two identical IgA or IgM heavy chains within at least one binding unit, or within at least two, at least three, at least four, at least five, or at least six binding units of the binding molecule comprise the heavy chain variable domain amino acid sequences as disclosed in Tables 2 and 3.

In certain aspects, the two light chains within the at least one binding unit of the binding molecule, or at least two, at least three, at least four, at least five, or at least six binding units of the binding molecule, are identical. In certain aspects, two identical light chains within at least one binding unit, or within at least two, at least three, at least four, at least five, or at least six binding units of the binding molecule are kappa light chains, e.g., human kappa light chains, or lambda light chains, e.g., human lambda light chains. In certain aspects, two identical light chains within at least one binding unit, or within at least two, at least three, at least four, at least five, or at least six binding units of the binding molecule each comprise the light chain variable domain amino acid sequences as disclosed in Tables 2 and 3.

In certain aspects at least one, at least two, at least three, at least four, at least five, or at least six binding units of a dimeric, pentameric, or hexameric binding molecule provided by this disclosure comprises or each comprise two identical IgA or IgM heavy chain constant regions each comprising identical heavy chain variable domain amino acid sequences as disclosed in Tables 2 and 3, and two identical light chains each comprising identical heavy chain variable domain amino acid sequences as disclosed in Tables 2 and 3. According to this aspect, the DR5 binding domains in the at least one binding unit of the binding molecule, or at least two, at least three, at least four, at least five, or at least six binding units of the binding molecule, can be identical. Further according to this aspect, a dimeric, pentameric, or hexameric binding molecule as provided herein can comprise at least one, at least two, at least three, at least four, at least five, at least six, at least seven, at least eight, at least nine, at least ten, at least eleven, or at least twelve copies of an DR5 binding domain as described above. In certain aspects at least two, at least three, at least four, at least five, or at least six of the binding units can be identical and, in certain aspects the binding units can comprise identical binding domains, e.g., at least two, at least three, at least four, at least five, at least six, at least seven, at least eight, at least nine, at least ten, at least eleven, or at least twelve DR5 binding domains can be identical.

In certain aspects, a dimeric, pentameric, or hexameric DR5 binding molecule as provided herein can possess advantageous structural or functional properties compared to other binding molecules. For example, the dimeric, pentameric, or hexameric DR5 binding relative to a corresponding bivalent binding molecule having the same antigen binding domains. Biological assays include, but are not limited to ELISA and Western blot caspase assays, and FACS analyses using stains indicative of apoptotic cell death such as annexin-v. In certain aspects a dimeric, pentameric, or hexameric binding molecule as provided herein can trigger apoptosis of a DR5-expressing cell at higher potency than an equivalent amount of a monospecific, bivalent IgG1 antibody or fragment thereof that specifically binds to the same DR5 epitope as the DR5 binding domain. In certain aspects a dimeric, pentameric, or hexameric binding molecule as provided herein can trigger apoptosis of a DR5-expressing cell at higher potency than an equivalent amount of monospecific, bivalent anti-DR5 monoclonal antibody or fragment thereof, where the antibody is, or comprises the same VH and VL regions as, the antibodies provided in Tables 2 and 3.

Polynucleotides, Vectors, and Host Cells

The disclosure further provides a polynucleotide, e.g., an isolated, recombinant, and/or non-naturally-occurring polynucleotide, comprising a nucleic acid sequence that encodes a polypeptide subunit of a dimeric, pentameric, or hexameric binding molecule as provided herein. By “polypeptide subunit” is meant a portion of a binding molecule, binding unit, or binding domain that can be independently translated. Examples include, without limitation, an antibody VH, an antibody VL, a single chain Fv, an antibody heavy chain, an antibody light chain, an antibody heavy chain constant region, an antibody light chain constant region, and/or any fragment thereof.

The disclosure further provides a composition comprising two or more polynucleotides, where the two or more polynucleotides collectively can encode a dimeric, pentameric, or hexameric binding molecule as described above. In certain aspects the composition can include a polynucleotide encoding an IgA or IgM heavy chain or fragment thereof, e.g., a human IgA or IgM heavy chain as described above where the IgA or IgM heavy chain comprises at least the VH of a DR5 binding domain, and a polynucleotide encoding a light chain or fragment thereof, e.g., a human kappa or lambda light chain that comprises at least the VL of a DR5 binding domain. A polynucleotide composition as provided can further include a polynucleotide encoding a J chain, e.g., a human J chain, or a fragment thereof or a variant thereof. In certain aspects the polynucleotides making up a composition as provided herein can be situated on two or three separate vectors, e.g., expression vectors. Such vectors are provided by the disclosure. In certain aspects two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, comprising a polynucleotide or two or more polynucleotides encoding a dimeric, pentameric, or hexameric DR5 binding molecule as provided herein, or any subunit thereof, a polynucleotide composition as provided herein, or a vector or two, three, or more vectors that collectively encode a dimeric, pentameric, or hexameric DR5 binding molecule as provided herein, or any subunit thereof. In certain aspects a host cell provided by the disclosure can express a dimeric, pentameric, or hexameric DR5 binding molecule as provided by this disclosure, or a subunit thereof.

In a related aspect, the disclosure provides a method of producing a dimeric, pentameric, or hexameric DR5 binding molecule as provided by this disclosure, where the method comprises culturing a host cell as described above and recovering the binding molecule.

Methods of Use

This disclosure provides a method for inhibiting, delaying, or reducing malignant cell growth in a subject with cancer by administering to the subject a combination therapy comprising an effective amount of a dimeric IgA antibody or a hexameric or pentameric IgM antibody, or a multimerized, antigen-binding fragment thereof that specifically and agonistically binds to DR5, wherein at least three antigen binding domains of the IgA or IgM antibody or fragment thereof are DR5-specific and agonistic, in combination with an effective amount of a chemotherapeutic agent, e.g., a DNA topoisomerase I inhibitor, e.g., irinotecan or topotecan, and/or a combination thereof, a nucleoside analog, e.g., Gemcitabine, cytosine arabinoside (ara-C), or fluorouracil (5-FU)). Exemplary anti-DR5 IgA and IgM antibodies and exemplary chemotherapeutic agents are described in detail elsewhere herein. In certain aspects, administration of the combination therapy provided herein can inhibit tumor or malignant cell growth partially or completely, can delay the progression of tumor and malignant cell growth in the subject, can prevent metastatic spread in the subject, can reduce the subject's tumor size, e.g., to allow more successful surgical removal, or can result in any combination of positive therapeutic responses in the subject. Exemplary therapeutic responses that can be achieved are described herein.

In certain aspects, administration of the combination therapy can result in enhanced therapeutic efficacy relative to administration of the anti-DR5 IgA and IgM antibody or the chemotherapeutic agent, e.g., the DNA topoisomerase I inhibitor, the nucleoside analog, or the pro-apoptotic agent, e.g., the BCL-2 inhibitor, alone. In certain aspects the improved treatment efficacy can be greater than the additive efficacy of each individual agent. In certain aspects the improved treatment efficacy over either agent administered alone, measured, e.g., in increased tumor growth delay (TGD), increased frequency of tumor regression, e.g., complete tumor regression, or increased survival is at least 5%, at least 10%, at least 20%, at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%. In certain aspects the improved treatment efficacy over the additive efficacy of both agents administered individually, measured, e.g., in increased tumor growth delay (TGD), increased frequency of tumor regression, e.g., complete tumor regression, or increased survival is at least 5%, at least 10%, at least 20%, at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%. In certain aspects the improvement can be complete tumor regression and full survival. The improved activity can, for example, allow a reduced dose to be used, or can result in more effective killing of cells that are resistant to killing by standard treatments. By “resistant” is meant any degree of reduced activity of “standard of care” for a given tumor or cancer type.

In certain aspects the combination treatment methods provided herein can facilitate cancer treatment, e.g., by slowing tumor growth, stalling tumor growth, or reducing the size of existing tumors, when administrated as an effective dose to a subject in need of cancer treatment.

In certain aspects the DR5-expressing cell is an immortalized cell line, e.g., a cancer cell. The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. (2006) Br. J. Cancer 94:1057-1065), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer including, but not limited to, squamous cell cancers, and cancers of mucinous origins, such as, mucinous ovarian cancer, cholangiocarcinoma (liver) and renal papillary carcinoma. Mucosal distribution, for example as provided by an IgA-based binding molecule as provided herein, could be beneficial for certain cancers, e.g., lung cancer, ovarian cancer, colorectal cancer, or squamous cell carcinoma.

Effective doses of compositions for treatment of cancer vary depending upon many different factors, including means of administration, target site, physiological state of the patient, whether the patient is human or an animal, other medications administered, and whether treatment is prophylactic or therapeutic. In certain aspects the treatment methods provided herein can provide increased safety, in that the composition exhibits greater cytotoxicity (e.g., induces apoptosis to a greater extent) on cancer cells than on non-cancer cells, e.g., normal human hepatocytes. Usually, the patient is a human, but non-human mammals including transgenic mammals can also be treated. Treatment dosages can be titrated using routine methods known to those of skill in the art to optimize safety and efficacy.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques.

The subject to be treated can be any animal, e.g., mammal, in need of treatment, in certain aspects, subject is a human subject.

In its simplest form, a preparation to be administered to a subject is a dimeric, pentameric, or hexameric binding anti-DR5 antibody as provided herein, or an antigen-binding, multimerizing fragment, variant, or derivative thereof, administered in conventional dosage form in combination with a chemotherapeutic agent, e.g., Irinotecan, where the agents can be combined with a pharmaceutical excipient, carrier or diluent as described elsewhere herein.

A DR5 binding molecule as provided herein or an antigen-binding, multimerizing fragment, variant, or derivative thereof can be administered by any suitable method as described elsewhere herein, e.g., via IV infusion. In certain aspects, a DR5 binding molecule as provided herein or an antigen-binding, multimerizing fragment, variant, or derivative thereof can be introduced into a tumor, or in the vicinity of a tumor cell.

All types of tumors are potentially amenable to treatment by this approach including, without limitation, carcinoma of the breast, lung, pancreas, ovary, kidney, colon and bladder, as well as melanomas, sarcomas and lymphomas. Mucosal distribution could be beneficial for certain cancers, e.g., lung cancer, ovarian cancer, colorectal cancer, or squamous cell carcinoma.

Topoisomerase I Inhibitors

Topoisomerases are popular targets for cancer chemotherapy, and a variety of inhibitors have been or are currently being developed. Compounds that inhibit type I topoisomerase are currently in use or are being developed as cancer chemotherapeutic agents. In particular, two derivatives of the natural type I topoisomerase inhibitor camptothecin, irinotecan (7-ethyl-10-[4-(1-piperidino)-1-piperidino]carbonyloxy-camptothecin, also called CPT-11), and topotecan (9-[(dimethylamino)Methyl]-10-hydroxy-(4S)-camptothecin, are currently marketed for the treatment of various cancers. Irinotecan is part of the “FOLFIRI” regimen of leucovorin calcium (calcium folinate), 5-fluorouracil, and Irinotecan widely used in the treatment of advanced-stage and metastatic colorectal cancer.

Irinotecan has the following formula:

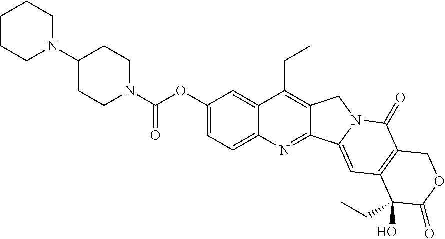 [SMILES: CCc1c2c(nc3ccc(OC(=O)N4CCC(N5CCCCC5)CC4)cc13)-c1cc3c(c(=O)n1C2)COC(=O)[C@]3(O)CC]

Topotecan has the following formula:

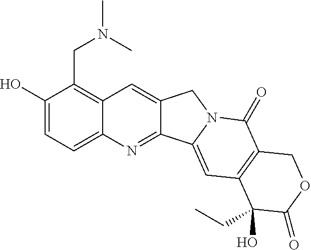 [SMILES: CC[C@@]1(O)C(=O)OCc2c1cc1n(c2=O)Cc2cc3c(CN(C)C)c(O)ccc3nc2-1]

Chemotherapeutic Nucleoside Analogs

Gemcitabine (2′,2′-difluoro 2′deoxycytidine, or dFdC) is a nucleoside analog used as chemotherapy. It is FDA approved for treatment of, e.g., breast, pancreatic, lung, and ovarian cancers. It has the following formula:

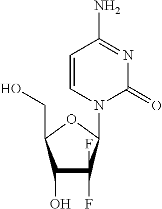

As a pyrimidine analog, the drug replaces one of the building blocks of nucleic acids in rapidly growing tumor cells, in this case cytidine, during DNA replication. The process arrests tumor growth, as new nucleosides cannot be attached to the “faulty” nucleoside, resulting in apoptosis (cellular “suicide”). Gemcitabine is used in various carcinomas: non-small cell lung cancer, pancreatic cancer, bladder cancer and breast cancer. Gemcitabine is the standard of care for many pancreatic cancers.

Other FDA-approved nucleoside analogs for cancer treatments include cytosine arabinoside (ara-C or Cytarabine) for treatment of acute myeloid leukemia (AML), acute lymphocytic leukemia (ALL), chronic myelogenous leukemia (CML) and non-Hodgkin's lymphoma (www_dot_drugs_dot_com/monograph/cytarabine.html (visited Nov. 14, 2018)), and fluorouracil (5-FU) for the treatment of colon cancer, esophageal cancer, stomach cancer, pancreatic cancer, breast cancer, basal cell carcinoma, and cervical cancer (www_dot_drugs_dot_com/monograph/fluorouracil.html (visited Nov. 14, 2018)). Ara-C has the following formula:

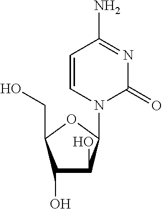

5-FU has the following formula:

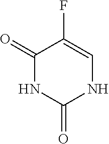

B-cell lymphoma-2 (BCL-2) Inhibitors

The BCL-2 family of proteins are associated with the regulation of cellular apoptosis. Proteins in this family associated with increased cell survival include BCL-2, BCL-X_{L}, BCL-2-like 2 (BCL-w), myeloid cell leukemia sequence 1 (MCL-1), and BCL-2-related protein A1 (BFL-1). Many small molecule inhibitors of these anti-apoptotic proteins have been studied as potential treatments for hematologic cancers such as relapsed or refractory chronic lymphoid leukemia (CLL) and acute myeloblastic leukemia (AML). See, e.g., Cang, S. et al., J. Hematol. Oncol. 8:129-136 (2015). Some of the first small molecule BCL-2 inhibitors developed were broad-spectrum. Id. For example, ABT-737 and ABT-263 (Navitoclax) inhibit the activity of BCL-2, BCL-X_{L}, and BCL-w. These drugs did not progress in clinical development because of low solubility and bioavailability of the former, and dose-limiting side effects of the latter. ABT-737 (4-{4-[(4′-Chloro-2-biphenylyl)methyl]-1-piperazinyl}-N-[(4-{[(2R)-4-(dimethylamino)-1-(phenylsulfanyl)-2-butanyl]amino}-3-nitrophenyl)sulfonyl]benzamide) has the following structure:

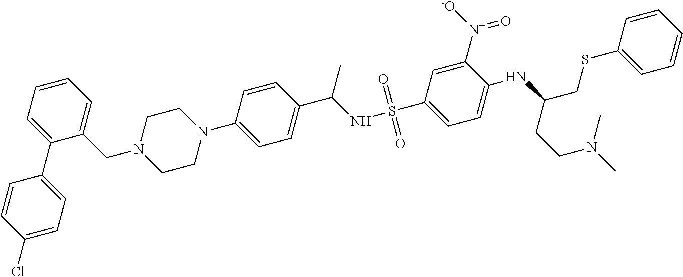

ABT-263 (Navitoclax; 4-(4-{[2-(4-Chlorophenyl)-5,5-dimethyl-1-cyclohexen-1-yl]methyl}-1-piperazinyl)-N-[(4-{[(2R)-4-(4-morpholinyl)-1-(phenylsulfany)-2-butanyl]amino}-3-[(trifluoromethyl)sulfonyl]phenyl)sulfonyl]benzamide) has the following structure:

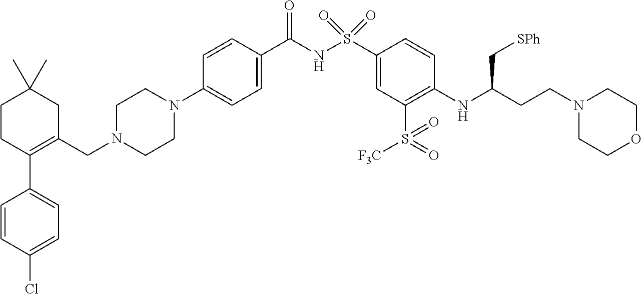

Venetoclax (ABT-199; 4-{4-[(4′-chloro-5,5-dimethyl[3,4,5,6-tetrahydro[1,1′-biphenyl]]-2-yl)methyl]piperazin-1-yl}-N-(3-nitro-4-{[(oxan-4-yl)methyl]amino}benzene-1-sulfonyl)-2-[(1H-pyrrolo[2,3-b]pyridin-5-yl)oxy]benzamide), on the other hand, selectively inhibits only the BCL-2 protein, and is currently indicated for treatment of CLL. Venetoclax has the following structure:

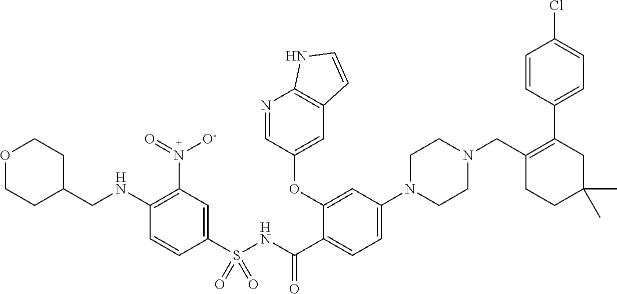

These drugs act to promote apoptosis of cancer cells, e.g., hematologic malignancies. Id.

Pharmaceutical Compositions and Administration Methods

Methods of preparing and administering a dimeric, pentameric, or hexameric TNF receptor binding molecule as provided herein to a subject in need thereof are well known to or are readily determined by those skilled in the art in view of this disclosure. The route of administration of a TNF receptor binding molecule can be, for example, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

As discussed herein, a dimeric, pentameric, or hexameric DR5 binding molecule as provided herein can be administered in a pharmaceutically effective amount for the in vivo treatment of cancers expressing DR5. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a dimeric, pentameric, or hexameric TNF receptor binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a dimeric, pentameric, or hexameric DR5 binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a dimeric, pentameric, or hexameric DR5 binding molecule as provided herein can be administered to a subject in need of therapy in an amount sufficient to produce a therapeutic effect. A dimeric, pentameric, or hexameric DR5 binding molecule as provided herein can be administered to the subject in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of a dimeric, pentameric, or hexameric DR5 binding molecule, that when administered brings about a positive therapeutic response with respect to treatment of a patient with cancer expressing DR5.

Therapeutically effective doses of the compositions disclosed herein for treatment of cancer can vary depending upon many different factors, including means of administration, target site, physiological state of the patient, whether the patient is human or an animal, other medications administered, and whether treatment is prophylactic or therapeutic. In certain aspects, the subject or patient is a human, but non-human mammals including transgenic mammals can also be treated. Treatment dosages can be titrated using routine methods known to those of skill in the art to optimize safety and efficacy.

The amount of a dimeric, pentameric, or hexameric DR5 binding molecule to be administered is readily determined by one of ordinary skill in the art without undue experimentation given this disclosure. Factors influencing the mode of administration and the respective amount of a dimeric, pentameric, or hexameric DR5 binding molecule include, but are not limited to, the severity of the disease, the history of the disease, and the age, height, weight, health, and physical condition of the individual undergoing therapy. Similarly, the amount of a dimeric, pentameric, or hexameric TNF receptor binding molecule to be administered will be dependent upon the mode of administration and whether the subject will undergo a single dose or multiple doses of this agent.

This disclosure also provides for the use of a dimeric, pentameric, or hexameric DR5 binding molecule in the manufacture of a medicament for treating, preventing, or managing cancer where the cancer expresses DR5.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

In the examples that follow, anti-DR5 IgM Mab #2, constructed as described in US Patent Application Publication No. 2018-0009897, and comprising the VH and VL amino acid SEQ ID NO: 5 and SEQ ID NO: 6 as provided in Table 2, and anti-DR5 IgM Mab #5, constructed as described in US Patent Application Publication No. 2018-0009897, and comprising the VH and VL amino acid SEQ ID NO: 7 and SEQ ID NO: 8 as provided in Table 2, were used. The anti-DR5 IgG used in the examples that follow likewise comprises the VH and VL amino acid sequences of SEQ ID NO: 5 and SEQ ID NO: 6 as provided in Table 2.

### Example 1: Anti-DR5 IgM and Irinotecan Combination Induces More Complete Tumor Cytotoxicity In Vitro

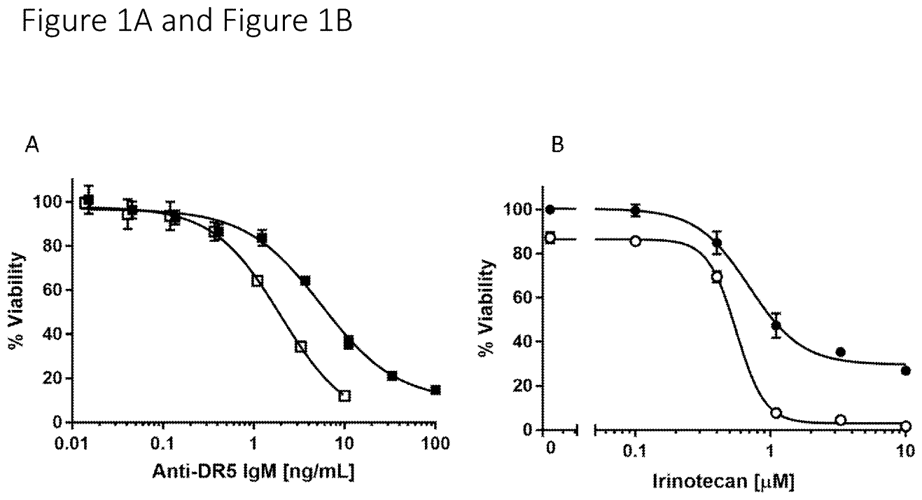

The in vitro efficacy of anti-DR5 IgM and Irinotecan combination therapy was assessed as follows. HCT15 tumor cells (2×10³ cells) were seeded and allowed to attach overnight. In the first experiment, cells were treated with Anti-DR5 IgM Mab #2 or Anti-DR5 IgM Mab #2 plus 0.4 μM of Irinotecan (Sigma 11406) for 72 hours at 37° C. Cell Titer Glo reagent (Promega G7572) was added and cell viability was read on a luminometer. The results are shown in FIG. 1A. In the second experiment, cells were treated with Irinotecan or Irinotecan plus 1 ng/mL of Anti-DR5 IgM Mab #2 for 72 hours at 37° C. Cell Titer Glo reagent was added and viability was read on a luminometer. The results are shown in FIG. 1B.

### Example 2: Anti-DR5 IgM and Irinotecan Combination Provides Significantly Enhanced Efficacy In Vivo

Combination therapy with an anti-DR5 IgM antibody and standard of care Irinotecan was assessed in an HCT15 tumor model as follows.

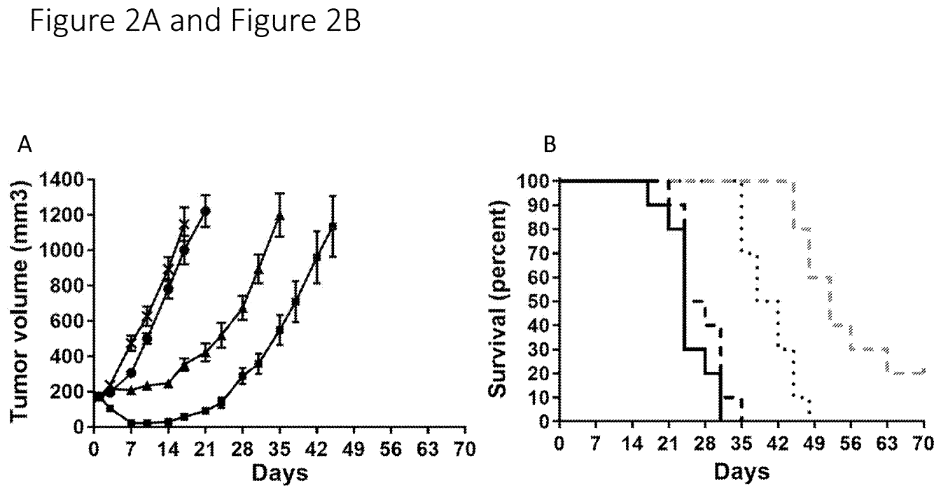

In the first experiment, anti-DR5 IgG, anti-DR5 IgM, and Irinotecan monotherapies were compared. Athymic nude mice were subcutaneously implanted with 1×10⁷ HCT15 tumor cells. When the tumors reached 150-200 mm³, the animals were dosed intravenously with either vehicle 5 times daily, 3 mg/kg of anti-DR5 IgG 3 times weekly, 3 mg/kg of anti-DR5 IgM Mab #2 5 times daily, or 80 mg/kg of Irinotecan (Sigma 11406) 3 times within the first week. Tumor volume (n=10 animals/group) is shown in FIG. 2A and overall survival is shown in FIG. 2B. The anti-DR5 IgM Mab #2 monotherapy was most effective.

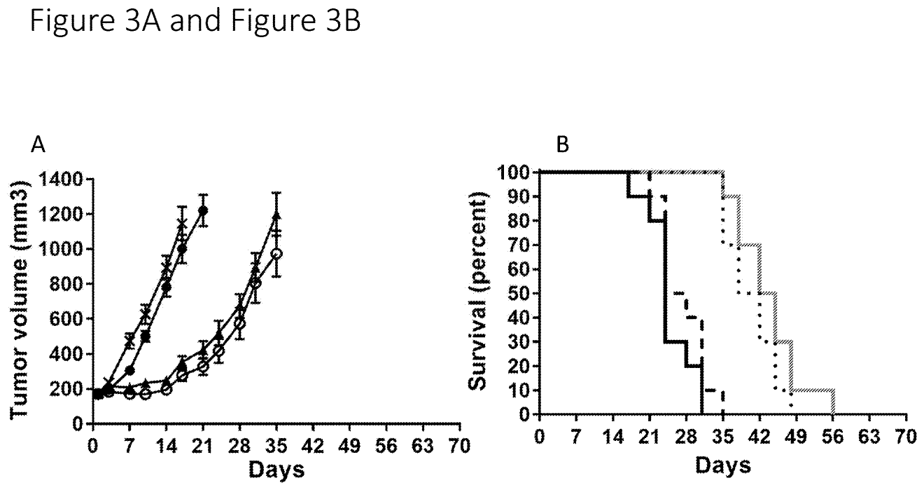

The second experiment demonstrates that combination therapy with an anti-DR5 IgG and Irinotecan provided little enhanced efficacy. Athymic nude mice were subcutaneously implanted with 1×10⁷ HCT15 tumor cells. When the tumors reached 150-200 mm³, the animals were dosed intravenously with either vehicle 5 times daily, 3 mg/kg of anti-DR5 IgG 3 times weekly, 80 mg/kg of Irinotecan (Sigma 11406) 3 times within the first week, or the combined anti-DR5 IgG and Irinotecan dosing regimens. Tumor volume (n=10 animals/group) is shown in FIG. 3A and overall survival is shown in FIG. 3B. The combination therapy provided only a slight enhancement over Irinotecan therapy alone.

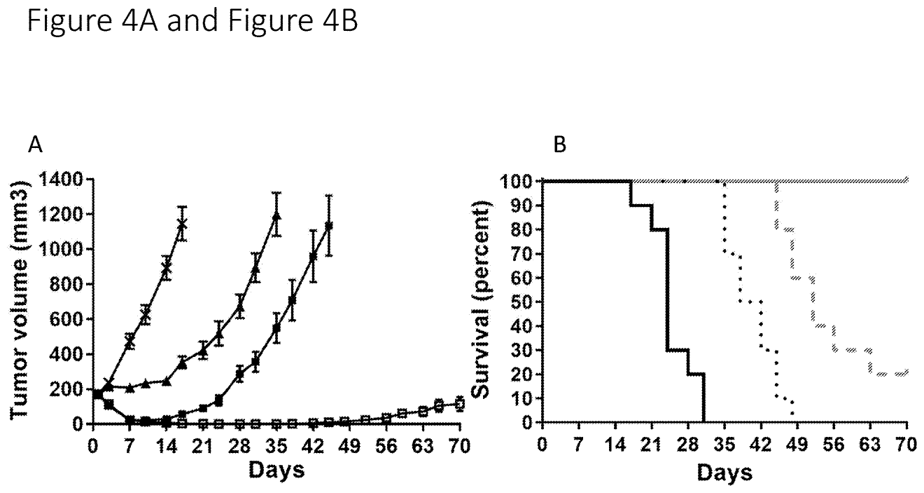

The third experiment demonstrates that combination therapy with an anti-DR5 IgM and Irinotecan can provide significant enhancement over standard of care. Athymic nude mice were subcutaneously implanted with 1×10⁷ HCT15 tumor cells. When the tumors reached 150-200 mm³, the animals were dosed intravenously with either vehicle 5 times daily, 3 mg/kg of anti-DR5 IgM 5 times daily, 80 mg/kg of Irinotecan (Sigma I1406) 3 times within the first week, or the combined anti-DR5 IgM and Irinotecan dosing regimens. Tumor volume (n=10 animals/group) is shown in FIG. 4A and overall survival is shown in FIG. 4B. The combination therapy resulted in tumor regression as well as complete survival out to 70 days.

### Example 3: Anti-DR5 IgM and Gemcitabine Combination Induces More Complete Pancreatic Tumor Cell Cytotoxicity In Vitro than Gemcitabine Alone

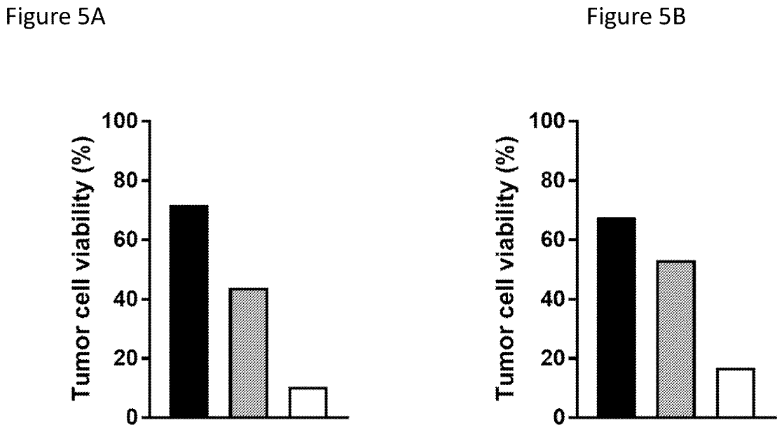

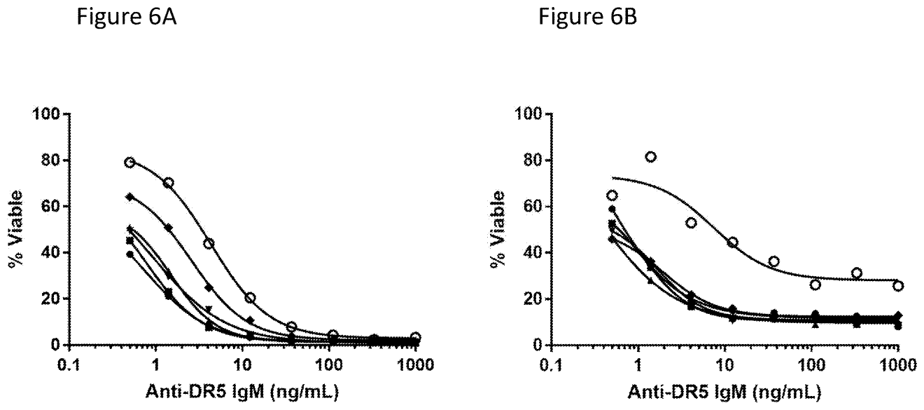

The in vitro potency of anti-DR5 IgM and Gemcitabine combination therapy in pancreatic tumor cell lines was assessed as follows. In the first experiment, 6×10³ BxPC3 tumor cells (ATCC CRL-1687) were seeded and allowed to attach overnight. Cells were treated with 4 ng/mL of anti-DR5 IgM Mab #5 (VH: SEQ ID NO: 7, VL SEQ ID NO: 8), 0.56 μM of Gemcitabine, or a combination of the two agents for 72 hours at 37° C. Cell Titer Glo reagent (Promega G7572) was added and cell viability was read on a luminometer. The results are shown in FIG. 5A. In the second experiment, 3×10³ Panc-1 tumor cells (ATCC CRL-1469) were seeded and allowed to attach overnight. Cells were treated with 4 ng/mL of anti-DR5 IgM Mab #5, 0.56 μM of Gemcitabine, or a combination of the two agents for 72 hours at 37° C. Cell Titer Glo reagent (Promega G7572) was added and cell viability was read on a luminometer. The results are shown in FIG. 5B. In the third experiment, 6×10³ BxPC3 tumor cells were seeded and the next day cells were treated with serial dilutions of anti-DR5 IgM Mab #5 and Gemcitabine in combination. After 72 hours at 37° C., Cell Titer Glo reagent (Promega G7572) was added and cell viability was read on a luminometer. The results are shown in FIG. 6A. In the fourth experiment, 3×10³ Panc-1 tumor cells were seeded and the next day cells were treated with serial dilutions of anti-DR5 IgM Mab #5 and Gemcitabine in combination. After 72 hours at 37° C., Cell Titer Glo reagent (Promega G7572) was added and cell viability was read on a luminometer. The results are shown in FIG. 6B.

### Example 4: Anti-DR5 IgM and Gemcitabine Combination Provides Enhanced Efficacy in an In Vivo Pancreatic Tumor Xenograft Model than Either Agent Alone or Combination Therapy with Anti-DR5 IgG

Combination therapy with an anti-DR5 IgM antibody and standard of care Gemcitabine was assessed in a BxPC3 pancreatic tumor model as follows.

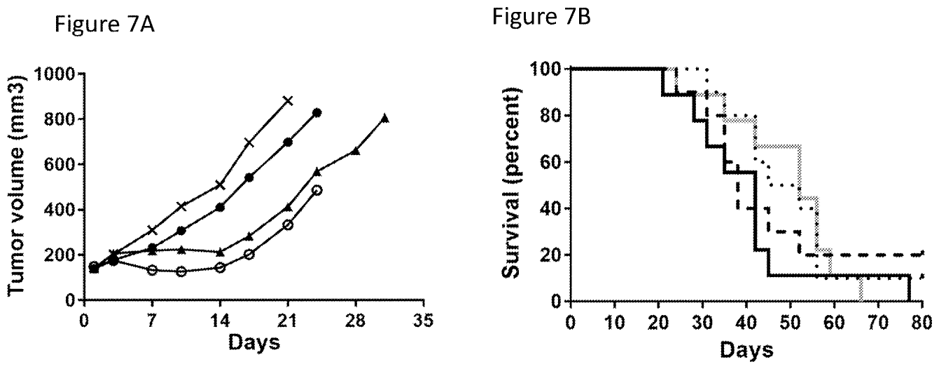

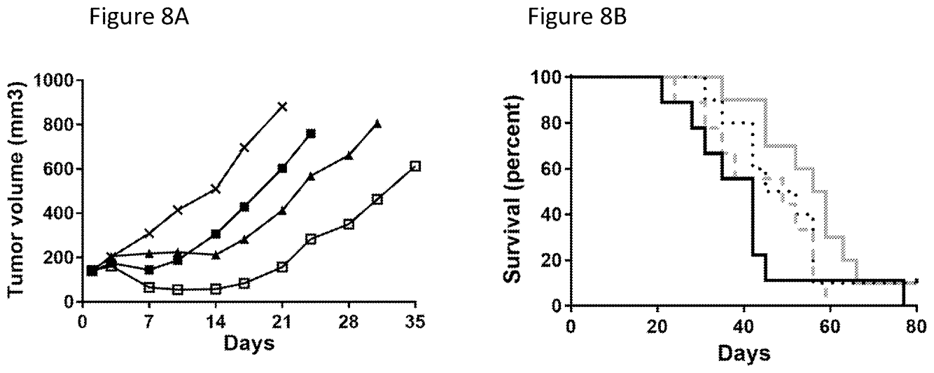

In the first experiment, nude mice were implanted subcutaneously with BxPC3 pancreatic tumor fragments. When the tumors reached 100-150 mm3, animals were dosed with either vehicle 7 times daily i.v., a single 3 mg/kg dose of anti-DR5 IgG Mab #2 i.v., 120 mg/kg of Gemcitabine every 3 days for a total of 4 doses i.p., or a combination of the anti-DR5 IgG and Gemcitabine dosing regimens. Tumor volume (n=9-10 animals/group) is shown in FIG. 7A and overall survival is shown in FIG. 7B. In the second experiment, nude mice were implanted subcutaneously with BxPC3 pancreatic tumor fragments. When the tumors reached 100-150 mm3, animals were dosed with either vehicle 7 times daily i.v., 3 mg/kg of anti-DR5 IgM Mab #2 7 times daily i.v., 120 mg/kg of Gemcitabine every 3 days for a total of 4 doses i.p., or a combination of the anti-DR5 IgM and Gemcitabine dosing regimens. Tumor volume (n=9-10 animals/group) is shown in FIG. 8A and overall survival is shown in FIG. 8B. The combination therapy with IgM and Gemcitabine provided reduced tumor volume relative to IgG combination therapy and relative any of the treatments alone.

### Example 5: Anti-DR5 IgM and Venetoclax (ABT-199) Combination Treatment Induces More Complete Cell Cytotoxicity In Vitro than Venetoclax Alone

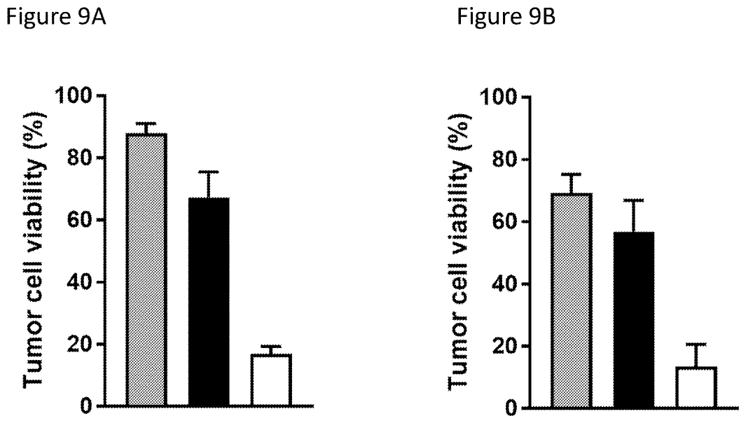

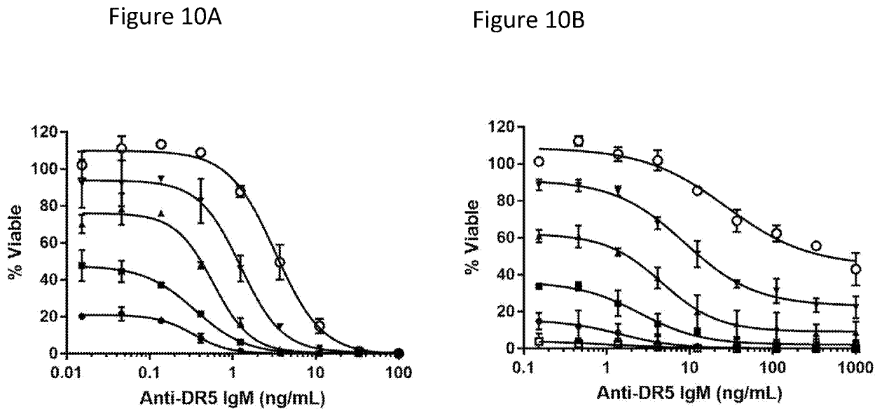

The in vitro potency of anti-DR5 IgM and Venetoclax combination treatment of AML cell lines was assessed as follows. In the first experiment, 3×10³ Molm-13 tumor cells (DSMZ ACC 554) were seeded and the next day cells were treated with 1.2 ng/mL of anti-DR5 IgM Mab #5, 3.7 nM of Venetoclax, or a combination of the two agents. After 72 hours at 37° C., Cell Titer Glo reagent (Promega G7572) was added and cell viability was read on a luminometer. The results are shown in FIG. 9A. In the second experiment, 3×10³ MV-4-11 tumor cells (ATCC CRL-9591) were seeded and the next day cells were treated with 37 ng/mL of anti-DR5 IgM Mab #5, 3.7 nM of Venetoclax, or a combination of the two agents. After 72 hours at 37° C., Cell Titer Glo reagent (Promega G7572) was added and cell viability was read on a luminometer. The results are shown in FIG. 9B. In the third experiment, 3×10³ Molm-13 tumor cells were seeded and the next day cells were treated with serial dilutions of anti-DR5 IgM Mab #5 and Venetoclax in combination. After 72 hours at 37° C., Cell Titer Glo reagent (Promega G7572) was added and cell viability was read on a luminometer. The results are shown in FIG. 10A. In the fourth experiment, 3×10³ MV-4-11 tumor cells were seeded and the next day cells were treated with serial dilutions of anti-DR5 IgM Mab #5 and Venetoclax in combination. After 72 hours at 37° C., Cell Titer Glo reagent (Promega G7572) was added and cell viability was read on a luminometer. The results are shown in FIG. 10B. The combination treatment showed an increase in killing of both cell lines over a range of antibody and drug concentrations.

What is claimed is:

1. A method for inhibiting, delaying, or reducing malignant cell growth in a subject with cancer, comprising administering to a subject in need of treatment a combination therapy comprising:
(a) an effective amount of a dimeric IgA antibody or a hexameric or pentameric IgM antibody, or a multimerized, antigen-binding fragment, variant, or derivative thereof that specifically and agonistically binds to DR5, wherein at least three antigen binding domains of the IgA or IgM antibody or fragment thereof are DR5-specific and agonistic; and
(b) an effective amount of a chemotherapeutic agent;

2. The method of claim 1, wherein the chemotherapeutic agent is a DNA topoisomerase I inhibitor.

3. The method of claim 2, wherein the DNA topoisomerase I inhibitor is a camptothecin derivative or an active variant, isomer, or salt thereof.

4. The method of claim 2, wherein the topoisomerase I inhibitor comprises Irinotecan or Topotecan.

5. The method of claim 4, wherein the topoisomerase I inhibitor comprises Irinotecan.

6. The method of claim 1, wherein the chemotherapeutic agent comprises a nucleoside analog or an active variant, isomer, or salt thereof.

7. The method of claim 6, wherein the nucleoside analog is Gemcitabine.

8. The method of claim 1, wherein the chemotherapeutic agent is a pro-apoptotic agent.

9. The method of claim 8, wherein the pro-apoptotic agent is a BCL-2 inhibitor or an active variant, isomer, or salt thereof.

10. The method of claim 9, wherein the BCL-2 inhibitor is Venetoclax.

11. The method of claim any one of claims 1 to 10, wherein at least three, at least four, at least five, at least six, at least seven, at least eight, at least nine, at least ten, at least eleven, or twelve antigen-binding domains of the antibody or fragment, variant or derivative thereof comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73 or the six CDRs with one or two amino acid substitutions in one or more of the CDRs.

12. The method of claim any one of claims 1 to 10, wherein at least three, at least four, at least five, at least six, at least seven, at least eight, at least nine, at least ten, at least eleven, or twelve antigen-binding domains of the antibody or fragment, variant or derivative thereof comprise an antibody VH and a VL, wherein the VH and VL comprise amino acid sequences at least 90% identical to SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or wherein the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

13. The method of claim 12, wherein at least four, at least ten, or twelve antigen-binding domains of the antibody or fragment, variant or derivative thereof comprise antibody VH and VL regions comprising the amino acid sequences SEQ ID NO: 1 and SEQ ID NO: 2, SEQ ID NO: 5 and SEQ ID NO: 6, SEQ ID NO: 7 and SEQ ID NO: 8, SEQ ID NO: 84 and SEQ ID NO: 85, or SEQ ID NO: 88 and SEQ ID NO: 89, respectively.

14. The method of any one of claims 1 to 13, wherein the antibody or fragment, variant or derivative thereof is a dimeric IgA antibody comprising two bivalent IgA binding units or fragments thereof and a J chain or fragment or variant thereof, wherein each binding unit comprises two IgA heavy chain constant regions or fragments thereof each associated with an antigen-binding domain.

15. The method of claim 14, wherein the IgA antibody or fragment thereof further comprises a secretory component, or fragment or variant thereof.

16. The method of claim 14 or claim 15, wherein the IgA heavy chain constant regions or fragments thereof each comprise a Cα1 domain, a Cα2 domain, and a Cα3-tp domain.

17. The method of any one of claims 14 to 16, wherein the IgA heavy chain constant region is a human IgA constant region.

18. The method of any one of claims 14 to 17, wherein each binding unit comprises two IgA heavy chains each comprising a VH situated amino terminal to the IgA constant region or fragment thereof, and two immunoglobulin light chains each comprising a VL situated amino terminal to an immunoglobulin light chain constant region.

19. The method of any one of claims 1 to 13, wherein the antibody or fragment, variant or derivative thereof is a pentameric or a hexameric IgM antibody comprising five or six bivalent IgM binding units, respectively, wherein each binding unit comprises two IgM heavy chain constant regions or fragments thereof each associated with an antigen-binding domain, and wherein the IgM heavy chain constant regions or fragments thereof each comprise a Cμ1 domain, a Cμ2 domain, a Cμ3 domain and a Cμ4-tp domain.

20. The method of claim 19, wherein the antibody or fragment, variant or derivative thereof is pentameric, and further comprises a J chain, or fragment thereof, or variant thereof.

21. The method of claim 19 or claim 20, wherein the IgM heavy chain constant region is a human IgM constant region.

22. The method of any one of claims 19 to 21, wherein each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

23. The method of any one of claims 1 to 22, wherein administration of the combination therapy results in enhanced therapeutic efficacy relative to administration of the antibody or fragment thereof or the chemotherapeutic agent alone.

24. The method of claim 23, wherein the enhanced therapeutic efficacy comprises a reduced tumor growth rate, tumor regression, or increased survival.

25. The method of any one of claims 1 to 24, wherein the subject is human.


SEQUENCE LISTING (89 sequences)

SEQ ID NO: 1 - protein, 121 aa, Artificial Sequence
  Description of Artificial Sequence: Synthetic polypeptide
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 2 - protein, 108 aa, Artificial Sequence
  Description of Artificial Sequence: Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 3 - protein, 121 aa, Artificial Sequence [sequence omitted]

SEQ ID NO: 4 - protein, 107 aa, Artificial Sequence [sequence omitted]

SEQ ID NO: 5 - protein, 122 aa, Artificial Sequence
  Description of Artificial Sequence: Synthetic polypeptide
QVQLQESGPGLVKPSQTLSLTCTVSGGSISSGDYFWSWIRQLPGKGLECIGHIHNSGTTYYNPSLKSRVTISVDTSKKQFSLRLSSVTAADTAVYYCARDRGGDYYYGMDVWGQGTTVTVSS

SEQ ID NO: 6 - protein, 108 aa, Artificial Sequence
  Description of Artificial Sequence: Synthetic polypeptide
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 7 - protein, 449 aa, Artificial Sequence
  Description of Artificial Sequence: Synthetic polypeptide
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Artificial Sequence
  Description of Artificial Sequence: Synthetic polypeptide
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 112 aa, Artificial Sequence
  Description of Artificial Sequence: Synthetic polypeptide
QIQLVQSGPELKKPGETVKISCKASGYTFTDFSMNWVKQAPGKGLKWMGWINTETGEPTYADDFKGRFALSMETSASTAYLQINNLKNEDTATYFCVRIDYWGQGTTLTVSS

SEQ ID NO: 10 - protein, 113 aa, Artificial Sequence
  Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPHTFGGGTKLEIKR

SEQ ID NO: 11 - protein, 145 aa, Artificial Sequence
  Description of Artificial Sequence: Synthetic polypeptide
MDWTWRILFLVAAATSAHSQVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSS

SEQ ID NO: 12 - protein, 128 aa, Artificial Sequence
  Description of Artificial Sequence: Synthetic polypeptide
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 13 - protein, 471 aa, Artificial Sequence
  Description of Artificial Sequence: Synthetic polypeptide
MKHLWFFLLLVAAPRWVLSEVQLQQSGPELVKPGASVKISCKASGYSFIGYFMNWMKQSHGKSLEWIGRFNPYNGDTFYNQKFKGKATLTVDKSSTTAHMELLSLTSEDSAVYFCGRSAYYFDSGGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 239 aa, Artificial Sequence
  Description of Artificial Sequence: Synthetic polypeptide
MVLQTQVFISLLLWISGAYGDVVMTQTPLSLPVSLGDQASISCRSSQSLVHSNGNTYLHWYLQKPGQSPKLLIYKVSNRFSGVPDRFSGSGSGTDFTLKISRVEAEDLGIYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 112 aa, Artificial Sequence
  Description of Artificial Sequence: Synthetic polypeptide
QVQLVQSGSELKKPGASVKVSCKASGYTFTDFSMNWVRQAPGQGLEWMGWINTETGEPTYADDFKGRFVFSLDTSVSTAYLQISSLKAEDTAVYYCARIDYWGQGTTVTVSS

SEQ ID NO: 16 - protein, 113 aa, Artificial Sequence
  Description of Artificial Sequence: Synthetic polypeptide
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCFQSTHVPHTFGQGTKLEIKR

SEQ ID NO: 17 - protein, 470 aa, Artificial Sequence
  Description of Artificial Sequence: Synthetic polypeptide
  MOD_RES (3)..(3): Any amino acid
MGXLGLSWVFLVVILEGVQCEVHLVESGGGLVRPGGSLKLSCAASGFAFSSYDMSWVRQTPEKRLEWVAYISDGGGITYYPDTMKGRFTISRDNAKNTLSLQMSSLKSEDTAMYYCARHITMVVGPFAYWGQGTLVTVSAASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPAPIEKTISKAKGQPREPQVYTLPPSREEMTKNQVSLTCLVKGFYPSDIAVEWESNGQPENNYKTTPPVLDSDGSFFLYSKLTMDKSRWQQGNVFSCSVMHEALHNHYTQKSLSLSPGK

SEQ ID NO: 18 - protein, 234 aa, Artificial Sequence
  Description of Artificial Sequence: Synthetic polypeptide
MRLPAQLLGLLMLWVSGSSGDIQMTQSSSSFSVSLGDRVTITCKASEDIYNRLAWYQQKPGNAPRLLISGATSLETGVPSRFSGSGSGKDYTLSITSLQTEDVATYYCQQYWSTPLTFGAGTKLELKRAVAAPSVDIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 466 aa, Artificial Sequence
  Description of Artificial Sequence: Synthetic polypeptide
MELGLSWVFLVVILEGVQCEVQLQQSGPELVKPGASVRMSCKASGYTFTSYFIHWVKQRPGQGLEWIGWIYPGNVNTKYSEKFKGKATLTADKSSSTAYMQFSSLTSEDSAVYFCARGEAGYFDYWGQGTTLTVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPAPIEKTISKAKGQPREPQVYTLPPSREEMTKNQVSLTCLVKGFYPSDIAVEWESNGQPENNYKTTPPVLDSDGSFFLYSKLTMDKSRWQQGNVFSCSVMHEALHNHYTQKSLSLSPGK

SEQ ID NO: 20 - protein, 234 aa, Artificial Sequence
  Description of Artificial Sequence: Synthetic polypeptide
MRLPAQLLGLLMLWVSGSSGDIVMTQSHKFMSTSVGDRVSITCKASQDVSTAVAWYQQKPGQSPRLLIYWASTRHTGVPDRFTGSGSGTDYTLTISSVQAEDQALYYCQQHYRTPWTFGGGTKLEIKRAVAAPSVDIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 121 aa, Artificial Sequence
  Description of Artificial Sequence: Synthetic polypeptide
QVQLVQSGAEVKKPGASVKVSCKASGYTFTSYDINWVRQATGQGLEWMGWMNPNSDNTGYAQKFQGRVTMTRNTSISTAYMELSSLRSEDTAVYYCARWNHYGSGSHFDYWGQGTLVTVSS

SEQ ID NO: 22 - protein, 107 aa, Artificial Sequence
  Description of Artificial Sequence: Synthetic polypeptide
DIQMTQSPSSLSASVGDRVTITCRASQSISIYLNWYQQKPGKAPKLLIYAASSLQSGVPLRFSGSGSGTDFTLTISSLQPEDIATYYCQQSYKTPLTFGGGTKVEIK

SEQ ID NO: 23 - protein, 120 aa, Artificial Sequence
  Description of Artificial Sequence: Synthetic polypeptide
QVQLQESGPGLVKPSQTLSLTCTVSGGSISSGGHYWSWIRQHPGKGLEWIGYIYYSGSTYYNPSLKSRVTISVDTSKNQFSLKLSSVTAADTAVYYCARDDSSGWGFDYWGQGILVTVSS

SEQ ID NO: 24 - protein, 107 aa, Artificial Sequence
  Description of Artificial Sequence: Synthetic polypeptide
DIQMTQSPSSLSASVGDRVTITCRASQGLRNDLGWFQQKPGKVTKRLIYAASSLQRGVPSRFSGSGSGTEFTLTISSLQPEDFATYYCLQHYSFPWTFGQGTKVEIK

SEQ ID NO: 25 - protein, 120 aa, Artificial Sequence
  Description of Artificial Sequence: Synthetic polypeptide
QVQLQESGPGLVKPSQTLSLTCTVSGGSISSGGHYWSWIRQHPGKGLEWIGYIYYSGSAYYNPSLKSRVTISVDTSKNQFSLKLSSVTAADTAVYYCARDDSSGWGFDYWGQGILVTVSS

SEQ ID NO: 26 - protein, 107 aa, Artificial Sequence
  Description of Artificial Sequence: Synthetic polypeptide
DIQMTQSPSSLSASVGDRVTITCRASQGLRNDLGWFQQKPGKAPKRLIYAASSLQRGVPSRFSGSGSGTEFTLTISSLQPEDFTTYFCLQHNSFPWTFGQGTKVEIK

SEQ ID NO: 27 - protein, 120 aa, Artificial Sequence; identical to SEQ ID NO: 25
  Description of Artificial Sequence: Synthetic polypeptide

SEQ ID NO: 28 - protein, 107 aa, Artificial Sequence; identical to SEQ ID NO: 26
  Description of Artificial Sequence: Synthetic polypeptide

SEQ ID NO: 29 - protein, 121 aa, Artificial Sequence
  Description of Artificial Sequence: Synthetic polypeptide
QVQLVESGGGLVKPGGSLRLSCAASGFTFSDYYMNWIRQAPGKGLEWVSHISSSGSILDYADSVKGRFTISRDNAKNSLYLQMNSLRVEDTAVYYCARDGAAAGTDAFDLWGQGTMVTVSS

SEQ ID NO: 30 - protein, 107 aa, Artificial Sequence
  Description of Artificial Sequence: Synthetic polypeptide
DIQMTQSPSSLSASVGDRVTITCRSSQSISNYINWYQQRPGKAPNLLIHDVSSFQSAVPSRFSRSGSGTVFTLTISSLQPEDFATYFCQQTYITPFTFGPGTKVDIK

SEQ ID NO: 31 - protein, 122 aa, Artificial Sequence
  Description of Artificial Sequence: Synthetic polypeptide
QVQLVESGGGVVQPGRSLRLSCAASGFTFSYYGIHWVRQAPGKGLEWVAVIWYDGSNKYYADSVKGRFTISRDNSKNTLYLQMNSLRAEDTAVYYCARGRYSSSSWWYFDLWGRGTLVTVSS

SEQ ID NO: 32 - protein, 107 aa, Artificial Sequence
  Description of Artificial Sequence: Synthetic polypeptide
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 33 - protein, 122 aa, Artificial Sequence
  Description of Artificial Sequence: Synthetic polypeptide
QVQAEQSGPGLVKPSETLSLTCTVSGGSISNYYWSWIRQPPGKGLEWIGYIYYSGSTKYNPSLKSRVTISVDTSKNQFSLKLTSVTTADTAVYYCARDSPRGFSGYEAFDSWGQGTLVTVSS

SEQ ID NO: 34 - protein, 113 aa, Artificial Sequence
  Description of Artificial Sequence: Synthetic polypeptide
DIVMTQSPDSLAVSLGERATINCKSSQSVLYRSNNKIYLAWYQQKPGQPPKLLIYWASTRESGVPDRFSGSGSGTDFTLTISSLLAEDVAVYYCQQYYSTPFTFGPGTKVDIK

SEQ ID NO: 35 - protein, 120 aa, Artificial Sequence
  Description of Artificial Sequence: Synthetic polypeptide
QVQLQESGPGLVKPSQTLSLTCTVSGGSISSDNYYWSWIRQHPGKGLEWIGYIYYSGSTYYNPSLKSRVTISVDTSKNQFSLKLSSVTAADTAVYYCARGVNWNFLFDIWGQGTMVTVSS

SEQ ID NO: 36 - protein, 112 aa, Artificial Sequence
  Description of Artificial Sequence: Synthetic polypeptide
DIVMTQSPLSLPVTPGEPASISCRSSQSLLRRNGYNYLDWYLQKPGQSPQLLIYLGSNRASGVPDRFSGSGSGTDFTLKISRVEAEDVGVYYCMQALQTPLTFGGGTEVEIK

SEQ ID NO: 37 - protein, 116 aa, Artificial Sequence
  Description of Artificial Sequence: Synthetic polypeptide
QVQLVESGGGLVKPGGSLRLSCAASGFTFSDYYMSWIRQAPGKGLEWVSYISRSGSTIYYADSVKGRFTISRDNAKNSLYLQMNSLRAEDTAVYYCARSLGGMDVWGQGTTVTVSS

SEQ ID NO: 38 - protein, 113 aa, Artificial Sequence
  Description of Artificial Sequence: Synthetic polypeptide
DIVMTQFPDSLAVSLGERATINCKSSQSVLHSSNNKNYLTWYQLKPGQPPKLLIYWASTRESGVPDRFSGSGSGTDFTLTISSLQAEDVAVYYCHQYYSTPSSFGQGTKLEIK

SEQ ID NO: 39 - protein, 129 aa, Artificial Sequence
  Description of Artificial Sequence: Synthetic polypeptide
QVQLVESGGGVVQPGRSLRLSCAASGFTFNNYGMHWVRQAPGKGLEWVAVIWYDGSNKYYADSVKGRFTISRDNSKNTLYLQMNSLRAEDTAVYYCARDRTVYSNSSPFYYYYYGMDVWGQGTTVTVSS

SEQ ID NO: 40 - protein, 107 aa, Artificial Sequence
  Description of Artificial Sequence: Synthetic polypeptide
DIQMTQSPSSLSASVGDRVTITCRTSQSISTYLNWYQQKPGKAPKLLISATSSLQSGVPSRFSGSGSGTDFTLTISSLQPEDFATYYCQQSYSTPLTFGGGTKVEIK

SEQ ID NO: 41 - protein, 129 aa, Artificial Sequence
  Description of Artificial Sequence: Synthetic polypeptide
QVQLVESGGGVVQPGRSLRLSCAASGFTFSTYGMHWVRQAPGKGLEWVAVIWYDGSNKYYADSVKGRFTISRDNSKNTLYLQMNSLRAEDTAVYYCARDRTVYSSSSPFYYYYYGMDVWGQGTTVTVSS

SEQ ID NO: 42 - protein, 107 aa, Artificial Sequence
  Description of Artificial Sequence: Synthetic polypeptide
DIQMTQSPSSLSASVGDRVTITCRASQSISSYLNWYQQKPGKAPKLLISATSSFQSGVPSRFSGSGSGTDFTLTISSLQPEDFAAYYCQQSYSTPLTFGGGTKVEIK

SEQ ID NO: 43 - protein, 119 aa, Artificial Sequence
  Description of Artificial Sequence: Synthetic polypeptide
QVQLQQWGARLLKPSETLSLTCAVYGGSFSGYYWSWIRQPPGKGLEWIGEINHSGSTNYNPSLKSRVTISVDTSKNQFSLKLRSVTAADTAVYYCARGGSSGYWYFDLWGRGTLVTVSS

SEQ ID NO: 44 - protein, 113 aa, Artificial Sequence
  Description of Artificial Sequence: Synthetic polypeptide
DIVMTQSPDSLAVSLGERATINCKSSQSVLHSSNNKNYLVWYQQKPGQPPKLLIYWASTRESGVPDRFSGSGSGTDFTLTISSLQAEDVAVYYCQQYYSTPLTFGGGTKVEIK

SEQ ID NO: 45 - protein, 121 aa, Artificial Sequence
  Description of Artificial Sequence: Synthetic polypeptide
EVQVVESGGGLVKPGGSLRLSCAASGFTFSSYSMNWVRQAPGKGLEWVSSISSSSSYIYYADSVKGRFTISRDNAKNSLYLQMNSLRAEDTAVYYCARGGSSWYGDWFDPWGQGTLVTVSS

SEQ ID NO: 46 - protein, 107 aa, Artificial Sequence
  Description of Artificial Sequence: Synthetic polypeptide
DIQMTQSPSSVSASVGDRVTITCRASQGISSWLVWYQQKPGKAPKLLIYAASSLQSGVPSRFSGSGSGTDFTLTISSLQPEDFATYYCQQANSFPFTFGGGTKVEIK

SEQ ID NO: 47 - protein, 126 aa, Artificial Sequence
  Description of Artificial Sequence: Synthetic polypeptide
QLVESGGGVVQPGRSLRLSCAASGFTFSSYGMHWVRQAPGKGLEWVAVIWYDGRNKYYADSVKGRFTISRDNSKNTLYLQMNSLRAEDTAVYYCAREVGYCTNGVCSYYYYGMDVWGQGTTVTVSS

SEQ ID NO: 48 - protein, 107 aa, Artificial Sequence
  Description of Artificial Sequence: Synthetic polypeptide
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 49 - protein, 122 aa, Artificial Sequence
  Description of Artificial Sequence: Synthetic polypeptide
QVQLQESGPGLVKPSQTLSLTCSVSGGSISSGGYYWSWIRQHPGKGLEWIGYIYYSGSTYCNPSLKSRVTISVDTSKNQFSLKLSSVTAADTAVYYCARDNGSGSYDWFDPWGQGILVTVSS

SEQ ID NO: 50 - protein, 107 aa, Artificial Sequence
  Description of Artificial Sequence: Synthetic polypeptide
DIQMTQSPSSVSASVGDRVTITCRASQGISSWLAWYQQKPGKAPKFLIFVASSFQSGVPSRFSGSGSGTDFTLTISSLQPEDFATYYCQQANSFPRTFGQGTKVEIK

SEQ ID NO: 51 - protein, 122 aa, Artificial Sequence
  Description of Artificial Sequence: Synthetic polypeptide
QVQMQESGPGLVKPSQTLSLTCTVSGGSISSGDYYWSWIRQHPGKNLEWIGYIYYSGSTYYNPSLKSRVTISVDTSKNQFSLKLSSVTAADTAVYYCARDNGSGSYDWFDPWGQGTLVTVSS

SEQ ID NO: 52 - protein, 107 aa, Artificial Sequence
  Description of Artificial Sequence: Synthetic polypeptide
DIQMTQSPSSVSASVGDRVTITCRASQGISSWLAWYQQKPGKAPKFLIFVASSLQSGVPSRFSGSGSGTDFTLTISSLQPEDFATYYCQQANSFPRTFGQGTKVEIK

SEQ ID NO: 53 - protein, 118 aa, Artificial Sequence
  Description of Artificial Sequence: Synthetic polypeptide
KVQLQQSGAELVKPGASVKLSCKASGYTFTDYTIHWVKQRSGQGLEWIGWFYPGGGYIKYNEKFKDRATLTADKSSNTVYMELSRLTSEGSAVYFCARHEEGIYFDYWGQGTTLTVSS

SEQ ID NO: 54 - protein, 109 aa, Artificial Sequence
  Description of Artificial Sequence: Synthetic polypeptide
DIAMTQSHKFMSTLVGDRVSITCKASQDVNTAIAWYQQKPGQSPKLLIYWASTRHTGVPDRFTGSGSGTDYTLTISSMEAEDAATYYCQQWSSNPLTFGAGTKLELKRA

SEQ ID NO: 55 - protein, 118 aa, Artificial Sequence
  Description of Artificial Sequence: Synthetic polypeptide
KVQLQQSGAELVKPGASVKLSCKASGYTFTDYTIHWVKQRSGQGLEWIGWFYPGGGYIKYNEKFKDRATLTADKSSNTVYMELSRLTSEDSAVYFCARHEEGIYFDYWGQGTTLTVSS

SEQ ID NO: 56 - protein, 104 aa, Artificial Sequence
  Description of Artificial Sequence: Synthetic polypeptide
DIVMTQSHKFMSTSVGDRVSITCKASQDVNTAIAWYQQKPGQSPKLLIYWASTRHTGVPDRFTGSGSGTDYTLTISSVQAEDLALYYCQQHYTTPFTFGSGTKL

SEQ ID NO: 57 - protein, 244 aa, Artificial Sequence
  Description of Artificial Sequence: Synthetic polypeptide
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 58 - protein, 245 aa, Artificial Sequence
  Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYHCARGGYSSSRSAAYDIWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 59 - protein, 246 aa, Artificial Sequence
  Description of Artificial Sequence: Synthetic polypeptide
QVQLVQSGAEVKKPGASVKISCEGSGYTFNSYTLHWLRQAPGQRLEWMGRINAGNGNTKYSQNFQGRLSITRDTSATTAYMELSSLRSEDTGVYYCARVFTYSFGMDVWGRGTLVTVSSGGGGSGGGGSGGGGSAQSVLTQPPSASGTPGQRVTISCSGGGSNIGRNSVSWYQQLPGTAPKLILYSNNQRPSGVPDRFSGSKSGTSASLAISGLRSEDEALYYCAAWDDSLSGGVFGGGTKLTVLG

SEQ ID NO: 60 - protein, 244 aa, Artificial Sequence
  Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVHRPGRSGYFDYWGRGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 61 - protein, 235 aa, Artificial Sequence
  Description of Artificial Sequence: Synthetic polypeptide
QVQLQQSGAEVKKPGASVRVSCQASGYSLSEYYIHWVRQAPGQGLEWMGWLNPNSGVTDYAQKFQGRVSMTRDTSISTAYMELSSLTFNDTAVYFCARGNGDYWGKGTLVTVSPGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCHSRDSSGWVFGGGTKLTVLG

SEQ ID NO: 62 - protein, 245 aa, Artificial Sequence
  Description of Artificial Sequence: Synthetic polypeptide
QVQLVQSGGGVVQPGRSLRLSCAASGFTFSPDAMHWVRQAPGKGLEWMGVISFDGSQTFYADSVKGRFTISRDNSQNTLYLQMNSLRSDDTAVYYCARAPARFFPLHFDIWGRGTMVTVSSGGGGSGGGGSGGGGSALSSELTQDPAVSVALGQTVRITCQGDSLRTHYASWYHQRPGRAPVLVNYPKDSRPSGIPDRFSGSSSGNTASLTIIGAQAADEGDYYCQSRDSSGVLFGGGTKVTVLG

SEQ ID NO: 63 - protein, 247 aa, Artificial Sequence
  Description of Artificial Sequence: Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDFSGYGDYLDYWGKGTLVTVSSGGGGSGGGGSGGGGSAQSALTQPPSASGSPGQSVTISCTGTSSDIGNYNYVSWYQQHPGKAPKLMIYEVNERPSGVPDRFSGSKSGNTASLTVSGLRPEDEADYYCSSYAGNNAVIFGGGTQLTVLG

SEQ ID NO: 64 - protein, 255 aa, Artificial Sequence
  Description of Artificial Sequence: Synthetic polypeptide
QVQLVQSGAEVKKPGASVKVSCKASGYTFTTHAMHWVRQAPGQSLEWMGWINTGNGNTKYSQSFQGRVSITRDTSANTAYMELSSLKSEDTAMYYCARASRDSSGYYYVPPGDFFDIWGQGTLVTVSSGGGGSGGGGSGGGGSAQSALTQPASVSGSPGQSITISCTGSRSDIGGYNFVSWYQQHPGKAPKLLIYDVYNRPSGISDHFSGSKSDNTASLTISGLQSEDDADYYCSSYAGYHTWIFGGGTKVTVLG

SEQ ID NO: 65 - protein, 248 aa, Artificial Sequence
  Description of Artificial Sequence: Synthetic polypeptide
EVQLVQSGAEVKKPGASVKLSCKASGYTLVNYFMHWVRQAPGQGPEWMGMINPSGGTTKNRQKFQDRVTMTRDTSTRTVYMELSGLTSEDTAVYYCATDFKGTDILFRDWGRGTLVTVSSGGGGSGGGGSGGGGSAQSVLTQPPSASGTPGQRVSISCSGSSSNIGSNTVIWYQQLPGTAPKLLMYSNDRRPSGVPDRFSGSKSGTSASLAISGLQSEDEADYYCATWDDSLNGHYVFGTGTKLTVLG

SEQ ID NO: 66 - protein, 243 aa, Artificial Sequence
  Description of Artificial Sequence: Synthetic polypeptide
QMQLVQSGGGLVKPGGSLRLSCAASGFTFSDYYMSWIRQAPGKGLEWVSAISGSGGSTYYADSVKGRFTISRDNSKNTLYLQMNSLRAEDTAVYYCARGGSTFDIWGRGTMVTVSSGGGGSGGGGSGGGGSAQPVLTQPPSASGTPGQRVTISCSGSNSNIGSRPVNWYQQLPGTAPKLLIQGNNQRPSGVPDRFSGSKSGTSASLAISGLQSEDEADYYCAAWDDSLTGYVFGPGTKLTVLG

SEQ ID NO: 67 - protein, 240 aa, Artificial Sequence
  Description of Artificial Sequence: Synthetic polypeptide
QMQLVQSGGAVVQPGRSLRLSCAASGFTFSSYGMHWVRQAPGKGLEWVAVISYDGSIKYYADSVKGRFTISRDNSKNTLYLQMNSLRAEDTAVYYCARERLRGLDPWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 68 - protein, 243 aa, Artificial Sequence
  Description of Artificial Sequence: Synthetic polypeptide
EVQLVETGGGLVQPGGSLRLSCAASGFTFSPYYMSWVRQAPGKGLEWVSAISGSGGSIYYADSVKGRFTISRDNSKNTLYLQMNSLRAEDTALYYCARGASGPDYWGRGTMVTVSSGGGGSGGGGSGGGGSAQSVLTQPPSVSAAPGQKVTISCSGSTSNIGNNYVSWYQQVPGTAPKLLIYDNNKRPSGIPDRFSGSKSGTSATLGITGLQTGDEADYYCGTWDSSLSALVFGGGTKVTVLG

SEQ ID NO: 69 - protein, 253 aa, Artificial Sequence
  Description of Artificial Sequence: Synthetic polypeptide
QVQLQQSGAEVKTPGSSVKVSCKASGGTFRNNAISWVRQAPGQGLEWMGGFIPKFGTTNHAQKFQGRVTMTADDSTNTVYMELSSLRSEDTAVYYCARGGAYCGGGRCYLYGMDVWGQGTLVTVSSGGGGSGGGGSGGGGSAQAVVIQEPSLTVSPGGTVTLTCGSSTGAVTSGHYPYWFQQKPGQAPRTLIYDTSNKRSWTPARFSGSLLGGKAALTLSGAQPEDEAEYYCLVSYSGSLVVFGGGTKLTVLG

SEQ ID NO: 70 - protein, 243 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKGAWLDYWGRGTMVTVSSGGGGSGGGGSGGGGSALNFMLTQPHSVSESPGKTVTISCTGSSGSVARNYVQWYQQRPGSAPTIVIYEDNRRPSGVPGRFSGSIDRSSNSASLTISGLQTEDEADYYCQSYNYNTWVFGGGTKLTVLG

SEQ ID NO: 71 - protein, 247 aa, Artificial Sequence
  Description of Artificial Sequence: Synthetic polypeptide
EVQLVQSGAEVKKPGASVKVSCRASGYTFTSYGITWVRQAPGQGLEWMGWISAYNGKTNYVQELQGRVTMTTDTSTSTVYMELTSLRSDDTAVYYCARRGNNYRFGYFDFWGQGTLVTVSSGGGGSGGGGSGGGGSALETTLTQSPGTLSLSPGERATLSCRASQSISSSNLAWYQQKPGRAPRLLIYGASSRAIGIPDRFSGSGSGTDFTLTISRLEAEDFAVYYCQQYGSSPITFGQGTRLEIKR

SEQ ID NO: 72 - protein, 248 aa, Artificial Sequence
  Description of Artificial Sequence: Synthetic polypeptide
QVQLQQSGPGLVKPSQTLSLTCAISGDSVSSTTVAWDWIRQSPSRGLEWLGRTYYRSKWYNEYAVSVKSRITINVDTSKNQISLQLNSVTPEDTAVYYCAREPDAGRGAFDIWGQGTTVTSPLRWGRFGWRGLGRGWLRSPVTQSPGTLSLSPGERATLSCRASQSVSSSHLAWYQQKPGQAPRLLIYGASSRATGIPDRFSGSGSGTDFTLTISSLEPEDFAVYYCQQRSNWPPRAVFGQGTRLEIK

SEQ ID NO: 73 - protein, 248 aa, Artificial Sequence
  Description of Artificial Sequence: Synthetic polypeptide
QVQLQQSGPGRVQPSQTLSLTCAISGDSVSNNNAAWYWIRQSPSRGLEWLGRTYYRSKWYNDYAVSVKSRITISPDTSKNQFSLQLNSVTPEDTAVYYCARRGDGNSYFDYWGQGTLVTVSSGILRWGRFGWRGLGRGWLEIVLTQSPGTLSLSPGERATLSCRASQSVSSGYVSWYRQKPGQAPRLLIYGASTRATGIPDRFSGSGSGTDFTLTISRLEPEDFAVYYCHQYGSSPNTYGQGTKVGIK

SEQ ID NO: 74 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 75 - skipped

SEQ ID NO: 76 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77 - skipped

SEQ ID NO: 78 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 79 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 80 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 81 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 82 - protein, 146 aa, Artificial Sequence
  Description of Artificial Sequence: Synthetic polypeptide
MDLMCKKMKHLWFFLLLVAAPRWVLSQLQLQESGPGLVKPSETLSLTCTVSGGSIISKSSYWGWIRQPPGKGLEWIGSIYYSGSTFYNPSLKSRVTISVDTSKNQFSLKLSSVTAADTAVYYCARLTVAEFDYWGQGTLVTVSSAS

SEQ ID NO: 83 - protein, 129 aa, Artificial Sequence
  Description of Artificial Sequence: Synthetic polypeptide
MEAPAQLLFLLLLWLPDTTGEIVLTQSPATLSLSPGERATLSCRASQSVSSFLAWYQQKPGQAPRLLIYDASNRATGIPARFSGSGSGTDFTLTISSLEPEDFAVYYCQQRSNWPLTFGPGTKVDIKRT

SEQ ID NO: 84 - protein, 146 aa, Artificial Sequence
  Description of Artificial Sequence: Synthetic polypeptide
MDLMCKKMKHLWFFLLLVAAPRWVLSQLQLQESGPGLVKPSETLSLTCTVSGGSISSRSNYWGWIRQPPGKGLEWIGNVYYRGSTYYNSSLKSRVTISVDTSKNQFSLKLSSVTVADTAVYYCARLSVAEFDYWGQGILVTVSSAS

SEQ ID NO: 85 - protein, 129 aa, Artificial Sequence
  Description of Artificial Sequence: Synthetic polypeptide
MEAPAQLLFLLLLWLPDTTGEIVLTQSPATLSLSPGERATLSCRASQSVSSFLAWYQQKPGQAPRLLIYDASNRATGSPARFSGSGSGTDFTLTISSLEPEDFAVYYCQQRSDWPLTFGPGTKVDIKRT

SEQ ID NO: 86 - protein, 154 aa, Artificial Sequence
  Description of Artificial Sequence: Synthetic polypeptide
MDLMCKKMKHLWFFLLLVAAPRWVLSQLQLQESGPGLVKPSETLSLTCTVSGGSISSSSYYWGWVRQPPGKGLEWIGSIHYSGSTFYNPSLKSRVTISVDTSKNQFSLKLSSVTAADTTVYYCARQGSTVVRGVYYYGMDVWGQGTTVTVSSAS

SEQ ID NO: 87 - protein, 131 aa, Artificial Sequence
  Description of Artificial Sequence: Synthetic polypeptide
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIKRT

SEQ ID NO: 88 - protein, 139 aa, Artificial Sequence
  Description of Artificial Sequence: Synthetic polypeptide
MEFGLSWLFLVAILKGVQCEVQLLESGGGLVQPGRSLRLSCAASGFTFSSYAMSWVRQAPGKGLEWVSAISGSGGSRYYADSVKGRFTISRDNSKNTLYLQMNSLRAEDTAVYYCAKESSGWFGAFDYWGQGTLVTVSS

SEQ ID NO: 89 - protein, 127 aa, Artificial Sequence
  Description of Artificial Sequence: Synthetic polypeptide
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPITFGQGTRLEIKR